(19) United States
(12) Patent Application Publication
(10) Pub. No.: US 2025/0352737 A1
(43) Pub. Date: Nov. 20, 2025

(54) SENSOR OR CANULA INSERTION PRE-TREATEMENT APPARATUS, METHOD AND SYSTEM

(71) Applicant: Michael Cameron, McKinney, TX (US)

(72) Inventor: Michael Cameron, McKinney, TX (US)

(21) Appl. No.: 18/779,034

(22) Filed: Jul. 21, 2024

Related U.S. Application Data
(60) Provisional application No. 63/647,487, filed on May 14, 2024.

(51) Int. Cl. A61M 5/42; A61M 5/142

(52) U.S. Cl. CPC A61M 5/422; A61M 5/14248

(57) ABSTRACT

A device and product to prepare and numb the skin of a user prior to the insertion of a sensor or canula under the skin. The invention is an applicator and one or more products dispensed thereby or disposed or positioned thereon, the applicator operable to apply a first product and in some cases, a second product, to the skin. The applicator is further operable to dry the skin so as to allow an adhesive backing of a device to be attached to the skin. Preferably, the invention is used to prepare and numb the skin prior to receiving a sensor from a continuous glucose monitor (CGM) or a canula from a insulin pump.

### CROSS REFERENCE TO RELATED APPLICATION

This application claims the benefit of, and incorporates herein by reference, U.S. Provisional Application No. 63/647,487, filed May 14, 2024, entitled DEVICE AND PRODUCTS, AND RELATED METHOD, FOR PREPARING AND DRYING THE EPIDERMIS PRIOR TO SENSOR OR CANULA INSERTION.

### BACKGROUND

### Insulin

Insulin is a hormone produced by the pancreas, a gland located behind the stomach. It plays a crucial role in regulating blood sugar (glucose) levels in the body. When one eats, carbohydrates are broken down into glucose and absorbed into the bloodstream. In response to rising blood glucose levels, the pancreas releases insulin into the bloodstream. Insulin acts as a “key” that unlocks cells, allowing them to take in glucose from the bloodstream. Once inside the cells, glucose is used as a source of energy for various cellular processes. Insulin stimulates the liver and muscle cells to take up excess glucose and store it in the form of glycogen. This helps regulate blood glucose levels by preventing them from rising too high after meals. Insulin also inhibits the liver from producing glucose, helping to prevent excessive glucose release into the bloodstream when blood sugar levels are already elevated. Insulin plays a role in regulating the metabolism of proteins and fats. It promotes the synthesis of proteins and inhibits the breakdown of protein into amino acids. Additionally, insulin promotes the storage of excess fats in adipose tissue.

In individuals with diabetes, there is either a deficiency of insulin (Type 1 diabetes) or a resistance to its effects (Type 2 diabetes), leading to elevated blood glucose levels. In Type 1 diabetes, the immune system mistakenly attacks and destroys the insulin-producing beta cells in the pancreas, resulting in little to no insulin production. In Type 2 diabetes, the body becomes resistant to the effects of insulin, and the pancreas may not produce enough insulin to overcome this resistance.

Management of diabetes often involves strategies to regulate blood glucose levels, including insulin therapy for individuals with Type 1 diabetes or those with Type 2 diabetes who require supplemental insulin. Insulin therapy aims to mimic the natural pattern of insulin secretion in response to meals and to maintain blood glucose levels within a target range to prevent complications associated with high or low blood sugar levels.

### Diabetes

Diabetes is a chronic metabolic disorder characterized by elevated levels of glucose (sugar) in the blood. This occurs either due to a deficiency of insulin, the hormone responsible for regulating blood sugar levels, or the body's inability to effectively use the insulin it produces. There are several types of diabetes, including Type 1 diabetes, Type 2 diabetes, gestational diabetes, and other less common forms.

Type 1 diabetes, also known as insulin-dependent diabetes or juvenile diabetes, typically develops in childhood or adolescence, although it can occur at any age. It is an autoimmune condition in which the body's immune system mistakenly attacks and destroys the insulin-producing beta cells in the pancreas. As a result, individuals with Type 1 diabetes produce little to no insulin and require lifelong insulin therapy to regulate their blood sugar levels.

Type 2 diabetes is the most common form of diabetes, accounting for the majority of cases worldwide. It usually develops in adults, although it is increasingly being diagnosed in children and adolescents due to rising rates of obesity and sedentary lifestyles. In Type 2 diabetes, the body becomes resistant to the effects of insulin, and the pancreas may not produce enough insulin to overcome this resistance. This leads to elevated blood sugar levels. Type 2 diabetes is often managed with lifestyle modifications, such as healthy eating, regular exercise, weight management, and medications, including oral glucose-lowering drugs and sometimes insulin therapy.

Gestational diabetes occurs during pregnancy and is characterized by elevated blood sugar levels that develop or are first recognized during pregnancy. It usually resolves after childbirth, but women who have had gestational diabetes have an increased risk of developing Type 2 diabetes later in life. Gestational diabetes can often be managed with dietary changes and exercise, but sometimes insulin therapy is needed to control blood sugar levels during pregnancy.

There are other, less common forms of diabetes, including monogenic diabetes, which results from mutations in a single gene, and secondary diabetes, which occurs as a result of another medical condition or medication.

Uncontrolled diabetes can lead to serious complications, including cardiovascular disease, kidney disease, nerve damage (neuropathy), eye damage (retinopathy), and foot problems that can lead to amputation. Therefore, early diagnosis, proper management, and lifestyle modifications are essential for preventing complications and maintaining overall health and well-being in individuals with diabetes.

### Blood Glucose Meter

A blood glucose meter is a medical device used to measure the concentration of glucose in the blood. It's a crucial tool for people with diabetes, as it allows them to monitor their blood sugar levels regularly. This monitoring helps individuals with diabetes manage their condition effectively by making informed decisions about diet, exercise, and medication.

The key components and evolution of blood glucose meters is as follows. A lancet device is used to prick the fingertip or another area to obtain a small drop of blood for testing. A small disposable test strip contains chemicals that react with glucose in the blood. A handheld meter is a device that reads the glucose level from the test strip and displays the result.

### Continuous Glucose Monitor (CGM)

CGM systems have emerged as an alternative or complementary technology to traditional blood glucose meters. CGM continuously monitors glucose levels throughout the day via a sensor inserted under the skin, providing real-time data and trends. Accuracy is paramount in blood glucose monitoring, as decisions about medication dosage, diet, and exercise are based on these readings. Manufacturers rigorously test their meters to ensure accuracy within acceptable limits. However, users should be aware of factors that can affect accuracy, such as improper use, environmental conditions, and calibration issues. Many modern blood glucose meters feature user-friendly interfaces, with large, easy-to-read displays and intuitive controls. Connectivity options, such as Bluetooth or USB, allow users to transfer data from the meter to a smartphone or computer for long-term tracking and analysis.

Overall, blood glucose meters play a vital role in diabetes management, empowering individuals to monitor and control their blood sugar levels effectively, thereby reducing the risk of complications associated with diabetes.

The CGM consists of three main components: a sensor, a transmitter, and a receiver or smartphone app. Here's how a CGM typically works. The process begins with the insertion of a small sensor under the skin, usually on the abdomen or upper arm. This sensor contains a tiny, flexible filament that is inserted just below the skin's surface. The insertion process can be painful and uncomfortable and is usually done by the user at home.

Once inserted, the sensor continuously measures glucose levels in the interstitial fluid, which surrounds the body's cells. The sensor uses a chemical reaction to detect glucose levels and convert them into an electrical signal. The sensor is connected to a small transmitter that is attached to the skin above the sensor. The transmitter collects the glucose data from the sensor and wirelessly transmits it to a receiver or a smartphone app. The receiver or smartphone app receives the glucose data transmitted by the transmitter. It displays real-time glucose readings, as well as trends and patterns in glucose levels over time. Some CGM systems also provide alerts for high or low glucose levels, helping users to take prompt action to manage their blood sugar levels. Depending on the CGM system, users may need to calibrate the device periodically by entering blood glucose readings obtained from a traditional fingerstick blood glucose meter. Calibration helps to ensure the accuracy of the CGM readings.

CGM systems typically store glucose data for several days to weeks, allowing users to review their glucose patterns and trends over time. Some CGM systems also offer data analysis features that can help users and healthcare providers identify patterns, adjust treatment plans, and make informed decisions about diabetes management. Overall, CGM technology provides valuable insights into glucose levels, helping individuals with diabetes to better understand and manage their condition, minimize the risk of hypo- and hyperglycemia, and improve long-term health outcomes.

Importantly; CGMs are retained on the user's skin using an adhesive material. The adhesive used on a CGM typically needs to be gentle on the skin while also providing secure adhesion to ensure the device stays in place for an extended period of time. Medical-grade acrylic adhesives are commonly used for CGM patches. These adhesives are designed to be hypoallergenic and water-resistant to withstand daily activities, showers, and sweat without causing irritation to the skin or losing adhesion prematurely. They offer a balance between gentle removal and strong adhesion to keep the CGM sensor securely attached to the skin throughout its wear period, which can range from several days to a week or more, depending on the specific CGM model.

### Insulin Pen

An insulin pen is a medical device used by diabetics to administer insulin. It is designed to deliver precise doses of insulin in a convenient and user-friendly manner. Insulin pens typically consist of the following components: The main body of the insulin pen houses the insulin cartridge, dose selector mechanism, and injection mechanism. The pen body is usually made of durable plastic and is designed to be easy to hold and use. A disposable cartridge contains insulin. The cartridge is inserted into the pen body and is pre-filled with a specific type and concentration of insulin. Insulin cartridges come in various sizes, depending on the brand and model of the pen. A dial or button on the pen body allows the user to select the desired dose of insulin. The dose selector typically has markings indicating the number of insulin units to be administered. A disposable, fine-gauge needle attaches to the tip of the pen for injection. Insulin pen needles are available in different lengths and thicknesses to accommodate individual preferences and injection sites. A button on the pen body is pressed to deliver the selected dose of insulin. Some insulin pens have a manual injection mechanism, while others feature an automatic injection mechanism that delivers the dose at the push of a button.

Insulin pens are compact and lightweight, making them easy to carry in a pocket or purse for use on the go. Insulin pens are simple to use and require minimal preparation before injection, making them suitable for individuals with limited dexterity or vision impairment. Insulin pens deliver precise doses of insulin, eliminating the need for manual measurement and reducing the risk of dosage errors. Insulin pens eliminate the need for carrying and disposing of vials and syringes, making insulin therapy more convenient and discreet. Overall, insulin pens are a convenient and practical option for insulin administration, providing flexibility and ease of use for individuals managing diabetes. However, the needle, when inserted into the skin, can be painful.

### Insulin Pump

An insulin pump is a medical device used by people with diabetes to deliver insulin continuously throughout the day. It is an alternative to multiple daily injections (MDI) of insulin and is primarily used by individuals with Type 1 diabetes, although some people with Type 2 diabetes may also benefit from insulin pump therapy.

The insulin pump is a small, battery-powered device about the size of a small cell phone or pager. It is worn externally, usually attached to a belt, pocket, or waistband, or carried in a pouch. The pump contains a reservoir that holds rapid-acting insulin. The pump is connected to the body via a thin, flexible tube called an infusion set. The infusion set consists of a cannula (a small, flexible plastic tube) that is inserted under the skin, typically on the abdomen or another fatty area. The cannula is attached to the pump via the infusion tubing, which delivers insulin from the pump to the body.

The insulin pump delivers a continuous, basal rate of insulin throughout the day and night, mimicking the normal pattern of insulin secretion by the pancreas. This basal rate can be adjusted and programmed according to the individual's insulin requirements, which may vary based on factors such as time of day, activity level, and metabolic needs.

In addition to basal insulin delivery, the insulin pump allows the user to administer bolus doses of insulin to cover meals or correct high blood sugar levels. Bolus doses can be delivered manually by entering the dose amount into the pump or calculated automatically based on pre-programmed insulin-to-carbohydrate ratios and correction factors. Insulin pumps are typically programmed using a user-friendly interface with buttons or a touchscreen display. Users can adjust basal rates, set temporary basal rates for activities or periods of increased insulin sensitivity, and enter blood glucose readings to calculate bolus doses. Many modern insulin pumps are also compatible with continuous glucose monitoring (CGM) systems, allowing for real-time monitoring of blood glucose levels and integration of CGM data into insulin dosing decisions.

Insulin pump therapy offers several advantages over traditional insulin injection therapy. Insulin pumps provide more precise insulin delivery and flexibility in dosing, which can help to optimize blood glucose control and reduce the risk of hypoglycemia (low blood sugar) and hyperglycemia (high blood sugar). Insulin pumps eliminate the need for multiple daily injections and offer greater flexibility in meal timing and activity level. Insulin pump settings can be tailored to the individual's lifestyle and insulin needs, allowing for personalized diabetes management.

However, insulin pump therapy also requires regular monitoring and adjustment of insulin doses, as well as careful attention to infusion site management and pump maintenance. Further, the insertion of the canula under the skin using the spring-loaded needle can be painful. Insulin pumps typically use similar adhesive materials to continuous glucose monitors (CGMs) to secure them to the skin. These adhesives are designed to be hypoallergenic, gentle on the skin, and water-resistant to ensure the pump stays securely attached during daily activities, including showers and exercise.

Medical-grade acrylic adhesives are commonly used for insulin pump patches, just like with CGMs. These adhesives offer a good balance between gentle removal and strong adhesion, allowing the pump to stay in place for several days before needing to be replaced. The adhesive must also be compatible with the materials used in the construction of the pump to ensure a secure and reliable bond.

### Medical Grade Adhesives

Medical-grade acrylic adhesives are formulated using a combination of specific materials and components to meet the requirements for medical applications. While the exact formulation can vary among manufacturers, here are some common materials and components used include the following.

Acrylic polymers are the primary component of medical-grade acrylic adhesives. These polymers provide the adhesive properties, such as tackiness and adhesion strength, needed for the adhesive to bond to the skin and medical devices. Plasticizers are additives used to modify the flexibility and softness of the adhesive. In medical-grade formulations, the choice of plasticizers is crucial to ensure skin-friendly properties and to minimize the risk of irritation or allergic reactions. Stabilizers are included to enhance the adhesive's stability and resistance to degradation over time, especially when exposed to environmental factors such as heat, light, or moisture. This helps maintain the adhesive's performance throughout its shelf life and during use. Crosslinking agents are compounds that promote the formation of chemical bonds within the adhesive polymer network. These bonds contribute to the adhesive's strength and durability, improving its resistance to shear forces and ensuring long-term adhesion performance. Solvents are used in the manufacturing process to dissolve and blend the adhesive components into a homogeneous mixture. They facilitate the application of the adhesive onto backing materials and aid in the removal of air bubbles during coating. Antimicrobial agents may be incorporated into medical-grade acrylic adhesives to help inhibit the growth of bacteria or fungi on the skin or device surface, reducing the risk of infection. Medical-grade acrylic adhesives are often coated onto backing materials such as films or nonwoven fabrics to form adhesive tapes or patches. These backing materials provide structural support, flexibility, and ease of handling during application and removal.

Disadvantageously the presence of a liquid such as an antiseptic or anesthetic can potentially interfere with the adhesion of the backing material to the skin. If the liquid anesthetic leaves behind residue on the skin after application, it can create a barrier between the skin and the adhesive, reducing its ability to form a strong bond. Residue can also affect the surface tension of the skin, making it more difficult for the adhesive to spread evenly and adhere effectively. Some liquid antiseptics or anesthetics contain water or other solvents that can increase the moisture content of the skin. Excessive moisture can weaken the adhesive bond by reducing its ability to form intimate contact with the skin surface. It can also cause the adhesive to swell or degrade over time, leading to premature detachment. Certain ingredients in the liquid anesthetic or antiseptic may react with components of the adhesive, altering its properties or causing it to degrade. For example, some anesthetics contain oils or emollients that can plasticize or soften the adhesive, affecting its tackiness and adhesion strength.

### Drying Agents

Drying agents can help expedite the drying process of liquids without significantly affecting their effectiveness. These agents are often referred to as “drying accelerators” or “drying enhancers.” They work by facilitating evaporation or absorption of moisture, thereby reducing the drying time while preserving the desired properties of the liquid. Common drying agents include the following. Ethanol or isopropyl alcohol is frequently used as a drying agent due to its rapid evaporation rate. Alcohol can help remove excess moisture from the skin surface without leaving residue behind. Alcohol-based products can cause skin irritation or dryness in some individuals. Antimicrobial powders such as cornstarch or talcum powder, have absorbent properties that can help absorb moisture and promote drying. These powders are often used in skincare products to absorb excess oil and moisture from the skin without interfering with the effectiveness of topical medications or treatments.

Silica gel is a desiccant material commonly used in packaging to absorb moisture and maintain dry conditions. It can also be used as a drying agent in topical formulations to help accelerate drying without affecting the formulation's properties. Certain clay minerals, such as kaolin or bentonite, have absorbent properties that can help draw out moisture from liquids and promote drying. These minerals are often used in skincare products, masks, and poultices to absorb excess oil and impurities from the skin.

Hygroscopic compounds, such as glycerin or propylene glycol, have the ability to attract and retain moisture from the surrounding environment. In some cases, these compounds can be used in combination with other drying agents to control moisture levels and promote faster drying of liquids.

Compressed air typically consists primarily of nitrogen (N2), oxygen (O2), and smaller amounts of other gases such as carbon dioxide (CO2), argon (Ar), and trace gases like neon (Ne), helium (He), and methane (CH4). Nitrogen is the most abundant component of compressed air, making up approximately 78% of Earth's atmosphere. It is chemically inert and non-reactive under normal conditions. Oxygen is the second most abundant component of compressed air, comprising approximately 21% of Earth's atmosphere. It is essential for respiration and combustion processes. Carbon dioxide is present in trace amounts in compressed air, typically less than 1%. It is a byproduct of respiration and combustion processes and plays a role in regulating Earth's climate. Argon is present in trace amounts in compressed air, typically less than 1%. It is an inert gas and does not react with other substances under normal conditions. Compressed air may also contain trace amounts of other gases such as neon (Ne), helium (He), methane (CH4), and hydrogen (H2), as well as water vapor (H2O) and airborne particulates.

Argon, in its pure form, is not considered dangerous to humans. It is a colorless, odorless, and non-toxic gas that makes up about 0.93% of Earth's atmosphere. Argon is chemically inert, meaning it does not react readily with other substances under normal conditions. However, argon, like other inert gases such as nitrogen and helium, can displace oxygen in poorly ventilated or confined spaces, leading to an oxygen-deficient atmosphere. Inhaling air with reduced oxygen levels can cause asphyxiation if oxygen levels drop below safe limits. Argon gas stored in pressurized containers, such as cylinders or tanks, can pose hazards if mishandled or if the containers are damaged. Rapid release of pressurized argon gas can cause explosions or ruptures, leading to potential injuries. Liquid argon, which has cryogenic properties and is extremely cold (−185.9° C. or −302.6° F.), can cause frostbite or cold burns if it comes into direct contact with the skin or tissues. While argon itself is not inherently dangerous, proper precautions should be taken when handling compressed or liquefied argon. Argon can be compressed. Like other gases, argon can be compressed into a smaller volume by increasing the pressure applied to it. This is a fundamental principle of gas behavior described by Boyle's Law, which states that the pressure of a gas is inversely proportional to its volume when the temperature remains constant. When argon gas is compressed, the molecules are forced closer together, resulting in an increase in pressure. This compressed argon can be stored in pressurized containers, such as cylinders or tanks.

Nitrogen, in its pure form, is not inherently dangerous to humans. In fact, nitrogen makes up about 78% of the Earth's atmosphere and is an essential component of the air we breathe. Nitrogen gas is non-toxic, colorless, odorless, and generally considered safe for human exposure in normal atmospheric concentrations. However, nitrogen gas can displace oxygen in poorly ventilated or confined spaces, leading to oxygen deficiency and asphyxiation if inhaled in high concentrations. Liquid nitrogen, which is extremely cold (−196° C. or −321° F.), can cause frostbite or cold burns if it comes into direct contact with the skin or tissues. Liquid nitrogen has cryogenic properties, meaning it can rapidly freeze materials upon contact. Accidental exposure to liquid nitrogen can cause severe tissue damage, especially if it splashes onto the skin or eyes. Nitrogen gas stored in pressurized containers, such as cylinders or tanks, can pose risks if mishandled or if the containers are damaged. Rapid release of pressurized nitrogen gas can cause explosions or ruptures, leading to potential injuries. Nitrogen gas itself is not dangerous under normal conditions but proper precautions should be taken when handling compressed or liquefied nitrogen to prevent accidents and ensure safety. This includes adequate ventilation, proper storage and handling procedures, and appropriate personal protective equipment when working with nitrogen in industrial or laboratory settings.

Nitrogen can be compressed. In fact, like many gases, nitrogen can be compressed into a smaller volume by increasing the pressure applied to it. This is a fundamental principle of gas behavior described by Boyle's Law, which states that the pressure of a gas is inversely proportional to its volume when the temperature remains constant. When nitrogen gas is compressed, the molecules are forced closer together, resulting in an increase in pressure. This compressed nitrogen can be stored in pressurized containers. Compressed nitrogen is commonly used in healthcare.

With respect to any compressed gas used as a drying agent, a mechanism is required to release the gas. To safely release compressed air, nitrogen or argon at high pressure while maintaining room temperature upon release, several precautions and techniques can be employed. A regulator and pressure gauge can be attached as a regulator to the compressed gas cylinder to control the flow rate and pressure of the gas being released. The regulator must be compatible with the gas and pressure rating of the cylinder. A pressure gauge is used to monitor the pressure inside the cylinder and adjust the regulator accordingly. The compressed gas cylinder should be stored in a controlled environment at room temperature (approximately 20-25° C. or 68-77° F.) to prevent temperature extremes that could affect the properties of the gas. It is key to have a mechanism that provides for a slow release of the gas. The valve on the regulator would be operative to release the gas slowly and steadily. The valve should be designed to avoid rapid or sudden releases of compressed gas, as this can cause temperature fluctuations and potential hazards. Rapid expansion of compressed gas can cause cooling effects due to the Joule-Thomson effect, leading to a decrease in temperature. To minimize temperature changes, release the gas slowly and control the flow rate using the regulator. Pressure-reducing devices, such as pressure-reducing valves or pressure regulators, downstream of the gas cylinder can be used to further reduce the pressure and ensure safe handling of the gas. The compressed gas should be released in a well-ventilated area to prevent the accumulation of gas and ensure proper dispersion.

### Absorbent Material

Several materials are available to use as a small absorbent pad that can be used to absorb liquids and dry the skin. Microfiber cloths are known for their excellent absorbency and durability. They can be reused multiple times without losing their effectiveness. Microfiber is highly absorbent, quick-drying, and durable. Microfiber is gentle on the skin if it's made from high-quality, soft microfibers. It's highly absorbent and quick-drying, making it suitable for use in reusable pads. Terry cloth is a highly absorbent fabric commonly used in towels. Cotton pads made from high-quality, tightly woven cotton can be reused many times while retaining their absorbency. Organic cotton is a popular choice for skin-related products due to its softness and hypoallergenic properties. It's gentle on the skin and highly absorbent, making it suitable for use in reusable pads. Linen is a strong and durable natural fiber. Bamboo fabric is known for its absorbency and antimicrobial properties. It is also known for its softness and moisture-wicking properties. It's gentle on the skin and has natural antimicrobial properties, which can help prevent bacteria growth. It's soft, breathable, and can hold a significant amount of liquid. Hemp fabric is known for its absorbent properties and durability. It's hypoallergenic and naturally resistant to mold and mildew, making it a good option for sensitive skin.

### Epidermis (e.g., Skin)

The epidermis, also referred to as the skin, is the outermost layer of the skin, serving as a protective barrier between the body's internal organs and the external environment. It is composed of multiple layers of cells that undergo constant turnover and renewal to maintain the integrity of the skin.

The stratum corneum is the outermost layer of the epidermis, consisting of dead, keratinized cells called corneocytes. These cells are continuously shed from the skin's surface and replaced by new cells from the underlying layers. The stratum corneum acts as a barrier to protect the body from environmental stressors, such as pathogens, chemicals, and UV radiation.

The stratum lucidum is a thin, translucent layer found only in thick skin, such as the palms of the hands and soles of the feet. It consists of a few layers of clear, flattened cells and helps to enhance the protective function of the skin.

The stratum granulosum is the layer of the epidermis located beneath the stratum lucidum (if present) or directly beneath the stratum corneum. It contains granular cells that produce keratin, a protein that provides structural support and waterproofing to the skin.

The stratum spinosum is the layer of the epidermis located beneath the stratum granulosum, consisting of several layers of living cells called keratinocytes. These cells are connected by desmosomes, protein structures that provide strength and flexibility to the skin.

The stratum basale (basal layer) is the deepest layer of the epidermis, in direct contact with the basement membrane that separates the epidermis from the dermis (the layer of skin beneath the epidermis). The stratum basale contains stem cells that continuously divide and differentiate into keratinocytes, replenishing the upper layers of the epidermis.

In addition to keratinocytes, the epidermis also contains other cell types, such as melanocytes which produce the pigment melanin responsible for skin color and Langerhans cells which are part of the immune system and help protect against infection. Overall, the epidermis plays a critical role in protecting the body from physical, chemical, and microbial threats, regulating water loss, and maintaining overall skin health.

### Nerves

The nerves associated with the epidermis are primarily sensory nerves, responsible for transmitting sensory information such as touch, temperature, pressure, and pain from the skin to the central nervous system (CNS), including the brain and spinal cord. These sensory nerves are located within the dermis, the layer of skin beneath the epidermis, and extend into the epidermal layers to innervate various regions of the skin.

The main types of sensory nerves found in the skin include the following: Mechanoreceptors are the sensory nerves that respond to mechanical stimuli, such as touch and pressure. There are different types of mechanoreceptors, including: Merkel cells which are found in the basal layer of the epidermis. These cells are associated with light touch and tactile discrimination. The Meissner's corpuscles are located in the dermal papillae of the dermis. These receptors are sensitive to light touch and vibration. The Pacinian corpuscles are found deeper in the dermis. These receptors detect deep pressure and vibration. Thermoreceptors are sensory nerves that respond to changes in temperature. Cold receptors (cold thermoreceptors) and warm receptors (warm thermoreceptors) are distributed throughout the skin to detect changes in environmental temperature and regulate body temperature accordingly.

Nociceptors are sensory nerves responsible for detecting noxious stimuli, such as pain. Nociceptors are activated by tissue damage or injury, chemical irritants, extreme temperatures, or mechanical pressure, signaling the brain to perceive pain and initiate protective responses.

These sensory nerves are interconnected with the nervous system, forming a complex network that allows for the perception and interpretation of sensory stimuli from the skin. The density and distribution of sensory nerves vary across different regions of the body, with some areas being more sensitive to touch or pain than others. Overall, the sensory nerves associated with the epidermis play a crucial role in the perception of touch, temperature, pressure, and pain, contributing to one's ability to interact with the external environment and maintain homeostasis.

### Topical Antiseptics

A topical antiseptic is a substance applied to the skin or mucous membranes to prevent or inhibit the growth of microorganisms, such as bacteria, fungi, and viruses. These antiseptics are commonly used to clean and disinfect minor cuts, scrapes, burns, and other wounds to prevent infection. Topical antiseptics work by destroying or inhibiting the growth of microorganisms on the skin's surface. They are typically applied directly to the affected area using solutions, gels, creams, sprays, or wipes. Some common types of topical antiseptics include:

Alcohol-Based antiseptics contain alcohol (such as ethyl alcohol or isopropyl alcohol) as the active ingredient. Alcohol-based antiseptics are effective against a broad spectrum of microorganisms and are commonly used for disinfecting skin before injections or minor surgical procedures. Iodine-Based antiseptics such as povidone-iodine (Betadine), contain iodine as the active ingredient. They have broad-spectrum antimicrobial activity and are effective against bacteria, fungi, and viruses. Iodine-based antiseptics are often used for wound cleansing and preoperative skin preparation. Chlorhexidine is a chemical antiseptic with broad-spectrum antimicrobial activity. It is commonly used as a skin cleanser in healthcare settings and for preoperative skin preparation. Chlorhexidine is available in various formulations, including solutions, gels, and wipes. Benzalkonium chloride is a quaternary ammonium compound with antimicrobial properties. It is commonly used as a preservative in topical antiseptic products, such as wound cleansers, hand sanitizers, and skin disinfectants. Hydrogen peroxide is an antiseptic agent with mild antimicrobial activity. It works by releasing oxygen, which helps to clean and disinfect wounds. Hydrogen peroxide is often used for cleaning minor cuts and scrapes. Topical antiseptics are an important part of wound care and infection prevention.

Alcohol-based antiseptics typically contain one or more types of alcohol as the active ingredient, along with other components such as water, emollients, and stabilizers. The most commonly used alcohols in alcohol-based antiseptics are ethanol (ethyl alcohol) and isopropyl alcohol (isopropanol). Here's a breakdown of the chemical composition of alcohol-based antiseptics: Ethanol (Ethyl Alcohol): Chemical Formula: C2H5OH. Ethanol is a colorless, volatile liquid alcohol with a characteristic odor. It is commonly used as a disinfectant and antiseptic due to its broad-spectrum antimicrobial activity, which includes bacteria, viruses, and fungi. Ethanol denatures proteins in microbial cell membranes, disrupting their structure and function, leading to cell death.

Isopropyl Alcohol (Isopropanol) has the chemical formula: C3H8O. Isopropyl alcohol is a colorless, flammable liquid alcohol with a slightly bitter taste and a characteristic odor. It is also commonly used as a disinfectant and antiseptic due to its antimicrobial properties. Isopropyl alcohol works similarly to ethanol by denaturing proteins in microbial cell membranes, leading to cell death. In addition to ethanol or isopropyl alcohol as the active ingredient, alcohol-based antiseptics may also contain other components. Alcohol-based antiseptics are typically solutions of alcohol and water, with alcohol concentrations ranging from 60% to 95%. Water helps to dilute the alcohol and improve its effectiveness as an antiseptic. Some alcohol-based antiseptics contain emollients or moisturizers, such as glycerin or aloe vera, to prevent drying and irritation of the skin. Stabilizers, such as hydrogen peroxide or glycerol, may be added to alcohol-based antiseptics to enhance their stability and shelf life.

Overall, alcohol-based antiseptics are effective for disinfecting the skin and preventing the spread of infectious microorganisms. They are commonly used for hand hygiene, wound cleansing, and surface disinfection in healthcare settings and household settings.

### Topical Numbing Formulations

Topical numbing creams or liquids, also known as topical anesthetics, typically contain active ingredients that temporarily desensitize the skin or mucous membranes, reducing the sensation of pain or discomfort. These products are commonly used to numb the skin before medical procedures, such as injections, vaccinations, minor surgeries, or cosmetic procedures. The specific ingredients in topical numbing creams or liquids can vary depending on the formulation, but some common active ingredients include Lidocaine and Benzocaine.

### Lidocaine

Lidocaine is one of the most commonly used topical anesthetics. It works by blocking nerve signals in the skin, temporarily numbing the area where it is applied. Lidocaine is available in various concentrations and formulations, including creams, gels, sprays, and patches. While some Lidocaine products require a prescription, many lower-concentration formulations are available for purchase without a prescription at pharmacies, drugstores, and online retailers. Common over-the-counter Lidocaine products include Lidocaine creams and gels. These topical formulations contain Lidocaine as the active ingredient and are designed to provide temporary relief from minor skin irritations, such as itching, burning, or pain associated with sunburns, insect bites, or minor cuts and scrapes. Lidocaine sprays are aerosolized formulations containing Lidocaine as the active ingredient. Lidocaine patches are adhesive patches containing a low concentration of Lidocaine that adhere to the skin and provide localized pain relief for conditions such as post-herpetic neuralgia (nerve pain following shingles), muscle or joint pain, or arthritis. Lidocaine lozenges or gargles contain Lidocaine as the active ingredient and are used to provide temporary relief from sore throat pain or discomfort.

Lidocaine is a local anesthetic commonly used to numb the skin or mucous membranes to relieve pain or discomfort during medical procedures. Its chemical composition is 2-(Diethylamino)-N-(2,6-dimethylphenyl) acetamide It's chemical formula is C14H22N2O. Lidocaine belongs to the amide class of local anesthetics. Lidocaine works by blocking nerve signals in the body, temporarily numbing the area where it is applied. It does so by inhibiting the function of voltage-gated sodium channels on the nerve membranes, preventing the transmission of pain signals to the brain. Lidocaine is available in various forms, including creams, gels, sprays, ointments, solutions, and patches, for topical application or injection. It is commonly used for procedures such as minor surgeries, dental procedures, skin biopsies, and epidural anesthesia. While lidocaine is generally considered safe when used as directed, it can cause side effects or allergic reactions in some individuals, such as skin irritation, burning sensation, or allergic contact dermatitis. Overuse or misuse of lidocaine-containing products can also lead to systemic side effects, such as dizziness, headache, or changes in heart rate.

### Benzocaine

Benzocaine is a topical anesthetic that works by blocking nerve signals in the skin. It is often used in over-the-counter numbing products for minor skin irritations and insect bites. Benzocaine is available in various forms, including creams, gels and sprays. Its chemical composition is as follows: Chemical Name: Ethyl 4-aminobenzoate Chemical Formula: C9H11NO2 Benzocaine belongs to the ester local anesthetic class. Benzocaine works by blocking nerve signals in the body, temporarily numbing the area where it is applied. It does so by inhibiting the function of voltage-gated sodium channels on the nerve membranes, preventing the transmission of pain signals to the brain. Benzocaine is available in various forms, including creams, gels, sprays, lozenges, and ointments, for topical application. It is also available in some combination products. While benzocaine is generally considered safe when used as directed, it can cause side effects or allergic reactions in some individuals, such as skin irritation, burning sensation, or allergic contact dermatitis. Overuse or misuse of benzocaine-containing products can also lead to systemic side effects, such as methemoglobinemia, a rare but serious condition that affects oxygen transport in the blood.

### Other Anesthetics

Tetracaine is a potent topical anesthetic that works by blocking nerve impulses in the skin. It is often used in combination with other local anesthetics for more prolonged numbing effects. Tetracaine is typically available in prescription formulations. Prilocaine is a local anesthetic similar to lidocaine, often used in combination with other numbing agents. It is commonly found in prescription formulations for topical anesthesia. EMLA cream is a topical anesthetic cream that contains a combination of lidocaine and prilocaine. It is commonly used to numb the skin before minor procedures, such as needle insertions, blood draws, or superficial skin surgeries. In addition to the active ingredients, topical numbing creams or liquids may also contain other ingredients such as emollients, preservatives, and stabilizers to improve the formulation's texture, stability, and shelf life. Lidocaine is available over the counter (OTC) in various formulations for topical use.

### Applicators

Portable applicators for applying a topical analgesic depend on personal preference, the type of analgesic, and the intended use. Stick applicators are convenient and mess-free, allowing for easy application of semi-solid formulations without the need for touching the affected area directly. Roll-on, aka rollerball applicators are convenient and mess-free, allowing for easy application of liquid formulations without the need for touching the affected area directly. Spray applicators are similar to rollerballs that provide a hands-free method of application of an aerosolized liquid that can cover larger areas quickly. Tube or pump dispensers allow for precise application to specific areas however these are not hands-free typically. They may also require some manual dexterity. Topical analgesic patches are convenient for targeted pain relief and can be considered portable but remain in place and provide long term infusion of a formulation.

A stick applicator typically consists of several components designed to contain, dispense, and apply a product effectively. The first step in making a stick-based product is formulating the product itself. This involves selecting and blending ingredients and thickeners to achieve the desired properties. The stick portion of the product is typically made from a mixture of active ingredients, waxes, oils, and other solidifying agents. This mixture is melted and poured into molds to form the stick shape. The molds may be cylindrical or oval-shaped, depending on the design of the final product. The stick is then allowed to cool and solidify. The stick is then packaged in suitable containers or tubes for retail sale. This may involve placing the stick in a twist-up or push-up container made of plastic or other materials. The container is often designed with a cap or lid to protect the product and prevent it from drying out. The final step involves labeling and branding the stick deodorant applicator with product information, usage instructions, and branding elements such as logos and graphics. This may be done through printing or labeling techniques. Throughout the manufacturing process, quality control measures are implemented to ensure that the stick product meets safety, efficacy, and regulatory standards. This includes testing the formulation for stability, performance, and microbiological safety, as well as conducting quality checks on the finished product to ensure proper assembly and packaging.

The packaging container for a stick product is typically made using injection molding or blow molding processes, both common methods in plastic manufacturing. The process begins with the preparation of the injection mold. The mold is usually made from metal and consists of two halves, which when closed together form the desired shape of the packaging container. Plastic resin pellets are heated until they reach a molten state. The specific type of plastic resin used depends on factors such as desired properties, cost, and compatibility with the product. The molten plastic resin is injected into the mold cavity under high pressure. The resin fills the cavity and takes on the shape of the mold. After injection, the mold is cooled to solidify the plastic and allow it to take on the shape of the mold cavity. Once the plastic has cooled and solidified, the mold is opened, and the newly formed packaging container is ejected from the mold. Any excess plastic is trimmed off, and additional finishing processes may be applied, such as printing, labeling, or surface treatment. For a blow molding process, the process begins with the extrusion of a tube-like plastic called a parison. The parison is formed by extruding molten plastic resin through a die. The parison is placed between two halves of a mold. The mold is closed, trapping the parison inside. Compressed air is injected into the parison, causing it to expand and take on the shape of the mold cavity. The plastic is forced against the walls of the mold, forming the desired shape of the packaging container. After the plastic has taken on the shape of the mold, it is cooled to solidify and set the shape. Once cooled, the mold is opened, and the newly formed packaging container is removed. Any excess plastic is trimmed off, and finishing processes may be applied as needed.

Both injection molding and blow molding are versatile processes that can be used to produce a wide range of packaging container shapes, sizes, and designs. The choice between the two methods depends on factors such as production volume, cost considerations, and the specific requirements of the packaging design.

A rollerball applicator refers to the container and mechanism used to dispense products, where the product is dispensed through a small rollerball attached to the tip of the applicator. The rollerball allows for smooth and controlled application of the product onto the skin. Rollerball applicators typically consist of several components, with the rollerball itself being a crucial part. The rollerball is made of materials that are compatible with the product being dispensed and provide smooth and even application.

Stainless steel is a popular choice for rollerball applicators due to its durability, corrosion resistance, and smooth surface finish. Stainless steel rollerballs provide excellent performance and are suitable for a wide range of cosmetic and personal care products. Some rollerball applicators feature rollerballs made of plastic materials, such as polypropylene (PP) or polyethylene terephthalate (PET). Plastic rollerballs are lightweight, cost-effective, and compatible with various formulations. They may be used for products where metal rollerballs are not suitable or cost prohibitive. In some cases, rollerball applicators may use glass rollerballs. Glass rollerballs provide a premium look and feel and are suitable for high-end cosmetic products or fragrances. Glass rollerballs are also inert and do not react with product formulations. Ceramic rollerballs are less common but can be found in some rollerball applicators. Ceramic rollerballs offer smooth and even application and may be used for specialized formulations or products requiring specific material properties.

Regardless of the material used, the rollerball is typically housed within a plastic or metal housing, which is attached to the top of the product container. The rollerball housing contains a cap or closure mechanism to prevent leakage and maintain product integrity when not in use.

Overall, the choice of material for the rollerball depends on factors such as product compatibility, performance requirements, and cost considerations. Manufacturers select materials that ensure optimal performance and user experience while meeting regulatory requirements for cosmetic and personal care products. An antiseptic liquid can be used in a rollerball applicator, provided that the rollerball and other components of the applicator are compatible with the liquid formulation. Rollerball applicators are commonly used for various cosmetic and personal care products, including antiseptic liquids, which are designed to disinfect and cleanse the skin. The rollerball material (such as stainless steel, plastic, glass, or ceramic) must be compatible with the antiseptic liquid formulation. Some liquids may react with certain materials, leading to degradation or corrosion of the rollerball or other components. Antiseptic liquids with low to moderate viscosity are typically suitable for rollerball applicators, as they allow for smooth and even dispensing through the rollerball. Liquids that are too thick or viscous may not flow easily through the rollerball, affecting application.

The stability of the antiseptic liquid formulation should be considered, particularly when exposed to air or light. Rollerball applicators typically feature a closure mechanism to prevent leakage and maintain product integrity, but formulations susceptible to degradation may require additional packaging or stability testing. The antiseptic liquid formulation must be safe for topical use on the skin and not contain any ingredients that may cause irritation or allergic reactions.

Examples of antiseptic liquids that may be suitable for use in rollerball applicators include alcohol-based antiseptics (e.g., ethanol or isopropyl alcohol solutions), chlorhexidine solutions, iodine-based antiseptics, and hydrogen peroxide solutions. These liquids are commonly used for wound cleansing, skin disinfection, and hand hygiene.

Using a liquid anesthetic formulation in a rollerball applicator may not be advisable due to several reasons. Rollerball applicators may not provide precise control over dosage and application, increasing the risk of overuse or accidental ingestion. Anesthetic formulations are typically liquid or gel-based and may not be well-suited for dispensing through a rollerball applicator. The viscosity and consistency of the liquid anesthetic may not flow smoothly or evenly through the rollerball, resulting in inconsistent application or clogging of the rollerball. Formulations containing an anesthetic must meet specific safety and efficacy standards, and the use of rollerball applicators may present additional challenges in ensuring proper dosage and application control.

### SUMMARY

The invention has a variety of aspects and embodiments. The objective of each aspect and embodiment is the use of the invention to prepare and numb the skin of a user prior to the insertion of a sensor or canula under the skin. The invention is an applicator and one or more products dispensed thereby, the applicator operable to apply a first product and in some cases, a second product, to the skin. The applicator is further operable to dry the skin so as to allow an adhesive backing of a device to be attached to the skin. Preferably, the invention is used to prepare the skin for receiving a CGM or insulin pump. In some aspects the first product is an antiseptic and the second product is an anesthetic, or vice versa, the first product is an anesthetic and the second product is an antiseptic. The drying aspect of the invention could be performed by the second product, such as a drying agent or antiseptic or it could be performed by the application of blown or compressed air from the applicator. Alternatively, the applicator could be a dual-ended stick or rollerball applicator, the applicator operable to dispense an antiseptic or anesthetic from a first end, the second end of the material having an absorbent material for drying the skin.

To those skilled in the art to which this invention relates, many changes in construction and widely differing embodiments and applications of the invention will suggest themselves without departing from the scope of the invention as defined herein. The disclosures and the descriptions herein are purely illustrative and are not intended to be in any sense limiting.

### FIGURES

For a better understanding of the present invention including the features, advantages and specific embodiments, reference is made to the detailed description along with accompanying Figures, wherein:

FIG. 1A is a top view of a single-ended stick applicator having a semi-solid or hard gel anesthetic half and a semi-solid or hard gel drying agent half;

FIG. 1B is top view of a single-ended stick applicator having a semi-solid or hard gel anesthetic half and a semi-solid or hard gel antiseptic half;

FIG. 1C is a top view of a single-ended stick applicator having a semi-solid or hard gel anesthetic product;

FIG. 1D is a front view of a single-ended stick applicator having a semi-solid or hard gel anesthetic product with the cap on;

FIG. 1E is a front view of a single-ended stick applicator having a semi-solid or hard gel anesthetic product with the cap removed;

FIG. 2A is a front view of a dual-ended stick applicator having a semi-solid or hard gel anesthetic product as a first product and an antiseptic or drying agent product as the second product with the caps on;

FIG. 2B is a front view of a dual-ended stick applicator having a semi-solid or hard gel anesthetic product as a first product and an antiseptic or drying agent product as the second product with the caps removed;

FIG. 2C is a front cutaway view of the dual-ended stick applicator;

FIG. 3A is a front view of a single-ended stick applicator with a compressed gas component appurtenant thereto with the cap on;

FIG. 3B is a front view of a single-ended stick applicator with a compressed gas component appurtenant thereto with the cap removed;

FIG. 3C is a side view of a single-ended stick applicator with a compressed gas component appurtenant thereto with the cap on;

FIG. 4A is a view of a dual-ended rollerball applicator having a viscous gel or liquid anesthetic product as a first product and a liquid or viscous gel drying agent product as the second product with the caps removed;

FIG. 4B is a view of a dual-ended rollerball applicator having a viscous gel or liquid anesthetic product as a first product and a liquid or viscous gel drying agent product as the second product with the caps on;

FIG. 5A is a view of a dual-ended rollerball applicator having a viscous gel or liquid anesthetic product as a first product and a liquid or viscous gel antiseptic product as the second product with the caps removed;

FIG. 5B is a view of a dual-ended rollerball applicator having a viscous gel or liquid anesthetic product as a first product and a liquid or viscous gel antiseptic product as the second product with the caps on;

FIG. 6 is a view of a single-ended rollerball applicator with a compressed gas component appurtenant thereto with the cap removed;

FIG. 7 is a view of a single-ended rollerball applicator having two chambers, one for holding an anesthetic and the other for holding an antiseptic or drying agent;

FIG. 8 is a flow chart of a method of the invention;

FIG. 9 is an adhesive back of an CGM or insulin pump having an anesthetic infused into the fabric mesh;

FIG. 10 is a view of a dual-ended stick applicator having a semi-solid or hard gel anesthetic product as a first product and a drying material as the second product with the caps removed; and

FIG. 11 is a view of a dual-ended rollerball applicator having a viscous gel or liquid anesthetic product as a first product and a drying material as the second product with the caps removed.

### DETAILED DESCRIPTION

While the making and using of the disclosed embodiments of the present invention is discussed in detail below, it should be appreciated that the present invention provides many applicable inventive concepts which can be embodied in a wide variety of specific contexts. Some features of the preferred embodiments shown and discussed may be simplified or exaggerated for illustrating the principles of the invention.

The invention has a variety of aspects and embodiments. In one aspect, the invention is an applicator in combination with a formulation of a fast-acting but short duration anesthetic and a drying material or formulation, in an aspect of the invention, suitable for use before applying a CGM or insulin pump to the skin.

The invention provides rapid onset of numbing or pain relief, but with a relatively brief duration of action. The fast-acting, short duration anesthetic can be one selected from Lidocaine, Benzocaine, Tetracaine, Prilocaine and EMLA Cream. Specifically, the anesthetic is formulated to dull the effectiveness of nociceptors. In an aspect, the formulation is designed to dry quickly so as not to interfere with the skin adhesion, biocompatibility, and long-term wearability of the adhesive backing of the CGM or insulin pump.

In an embodiment, the applicator of the invention is a twist-up or push-up stick applicator and the anesthetic is in a semi-solid or hard gel form and in another embodiment, the applicator of the invention is a rollerball applicator and the anesthetic is in liquid or viscous gel form. In a further embodiment, the applicator of the invention is a dual-ended stick applicator and the anesthetic is in a semi-solid or hard gel form and exposed from a first end. The second end of the dual-ended applicator exposes a semi-solid or hard gel drying agent or anesthetic or exposes an absorbent material for drying the applied anesthetic.

In yet a further embodiment, the applicator of the invention is a dual-ended rollerball applicator having a first end with a first rollerball and a second end with a second rollerball. The anesthetic is dispensed from the first rollerball and is in a liquid or viscous gel form and an antiseptic or drying agent is dispensed from the second rollerball. In yet a further embodiment, an anesthetic is dispensed from the first rollerball and is in a liquid or viscous gel form and the second end of the dual-ended applicator exposes an absorbent material for drying the applied anesthetic.

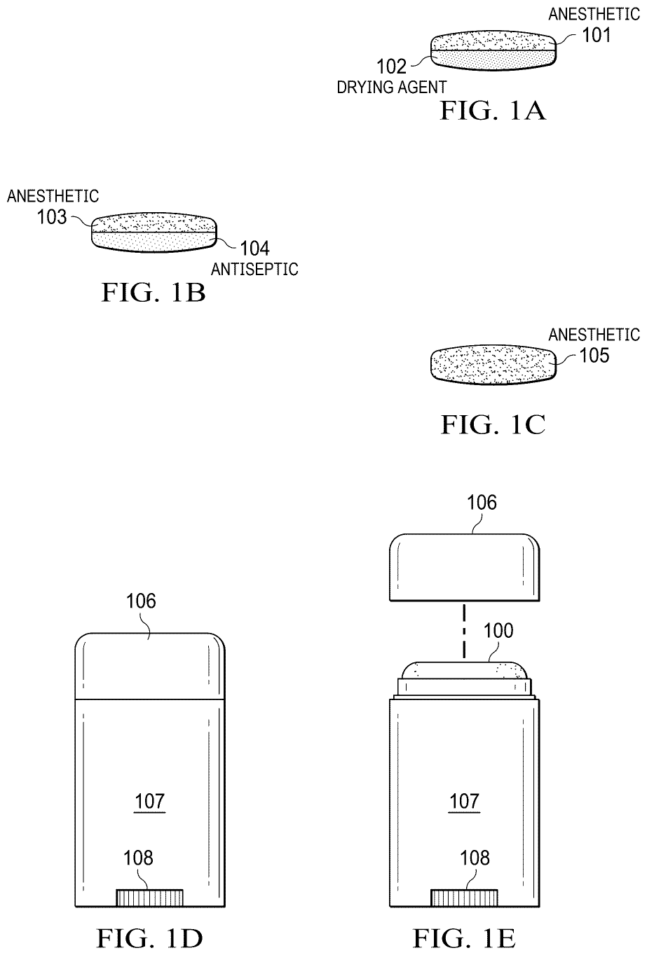

FIG. 1A is a top view of a product in the form of a stick (being referred to herein as the “product” or “stick”) to be disposed in a single-ended stick applicator, the stick being a semi-solid or hard gel anesthetic half 101 and a semi-solid or hard gel drying agent half 102. As seen therein, the semi-solid or hard gel anesthetic half 101 is separately contained within a lengthwise slice of the stick and the semi-solid or hard gel drying agent half 102 is contained within the other portion of the stick lengthwise. For example, a top half slice comprises the anesthetic and the bottom half lengthwise portion of the stick comprises an antiseptic. In this manner, when the stick is exposed from the top of the single stick applicator container and applied, both sides of the stick are applied to the skin.

FIG. 1B is top view of a product in the form of a stick to be disposed in a single-ended stick applicator, the stick being a semi-solid or hard gel anesthetic half 103 and a semi-solid or hard gel antiseptic half 104. FIG. 1C is a top view of a of a stick to be disposed in a single-ended stick applicator, being single-ended stick applicator having a semi-solid or hard gel anesthetic product 105.

FIG. 1D is a front view of a single-ended stick applicator 100 having a container body 107, a twist knob 108 and a cap 106. A semi-solid or hard gel anesthetic product is contained within the container body 107.

FIG. 1E is a front view of a single-ended stick applicator 100 having a container body 107, a twist knob 108 and a cap 106. The cap 106 is removed to show a semi-solid or hard gel anesthetic 109. The single-ended stick applicator can be an oblong shape or a cylindrical shape.

As seen in FIGS. 1A-1E, the invention comprises a stick applicator 100 having a semi-solid product 109 contained therein, the product 109 preferably being a fast acting but short duration anesthetic suitable for application before applying a CGM or insulin pump to the skin. In a further aspect, the product 109 is an antiseptic. In a further aspect, the product is a half lengthwise of anesthetic and a half lengthwise of an antiseptic. In a further aspect, the product is a half lengthwise anesthetic and a half lengthwise of a drying agent. In each of the foregoing, the anesthetic is one selected from Lidocaine, Benzocaine, Tetracaine, Prilocaine and EMLA Cream and the antiseptic is one selected from ethyl alcohol, isopropyl alcohol, povidone-iodine (Betadine), Chlorhexidine, Benzalkonium Chloride and Hydrogen Peroxide. Specifically, the anesthetic is formulated to dull the effectiveness of nociceptors. The semi-solid formulation is formulated to dry quickly so as not to interfere with the skin adhesion, biocompatibility, and long-term wearability of the adhesive backing of the CGM or insulin pump. In a further aspect, the product is in an oblong or elliptical form factor and in another aspect it is in a form factor of a cylindrical stick.

The invention further is an applicator having a gel formulation of a fast-acting, short duration anesthetic suitable for application before applying a CGM or insulin pump to the skin. The gel formulation is formulated to dry quickly so as not to interfere with the skin adhesion, biocompatibility, and long-term wearability of the adhesive backing of the CGM or insulin pump. The fast-acting, short duration anesthetic is one selected from Lidocaine, Benzocaine, Tetracaine, Prilocaine and EMLA Cream.

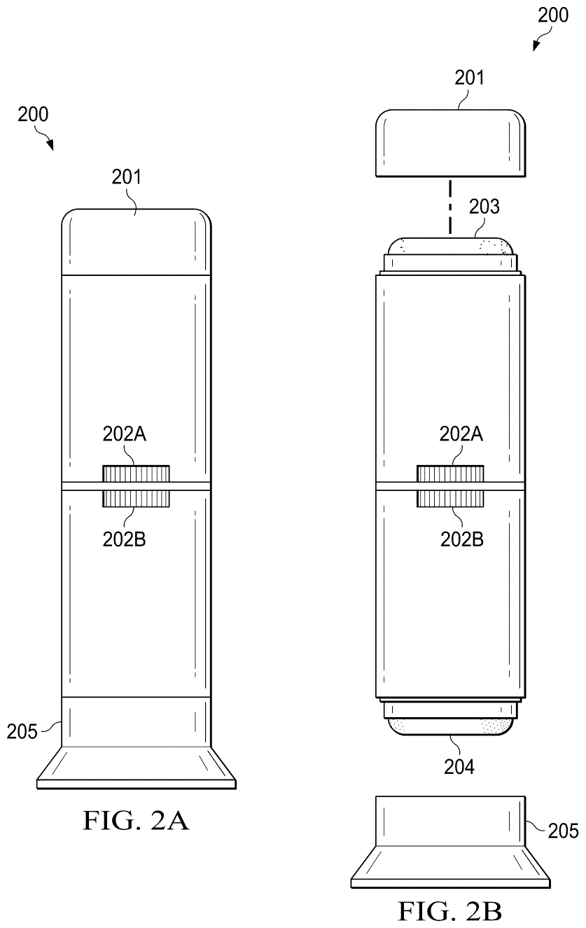

FIG. 2A is a front view of a dual-ended stick applicator 200 having a semi-solid or hard gel anesthetic product as a first product and an antiseptic or drying agent product as the second product with the caps 201, 205 on. As seen therein, cap 205 has a flared base so as to permit the invention 200 to be stored in an upright or vertical position. As further seen therein and in FIG. 2C, dual-ended stick applicator 200 has a first chamber and a second chamber and a first end in communication with the first chamber and a second end in communication with the second chamber.

In an embodiment, the first end has an opening for exposing a first product and the second end has an opening for exposing a second product. In such embodiment, each end is operable to expose a stick or product. The invention has a first twist knob 202A coupled to a twist mechanism, that when rotated, causes the movement of a first platform on which the first product is disposed, thereby exposing or retracting the first product from the first chamber. The first twist knob 202A is proximate to, and just offset from, the center of the dual-ended stick applicator 200. The dual-ended stick applicator can be an oblong shape or a cylindrical shape.

FIG. 2B is a front view of a dual-ended stick applicator 200 having a semi-solid or hard gel anesthetic product comprising the first product 203 and an antiseptic or drying agent product comprising the second product 204 with the caps 201, 205 removed.

FIG. 2C is a front cutaway view of the dual-ended stick applicator 200. The first twist mechanism comprises first twist knob 202A coupled to a first screw 206A that is rotatably coupled to the first platform 207A on which the first product 203 is disposed, thereby exposing or retracting the first product from the first chamber. The second twist mechanism comprises second twist knob 202B that is rotatably coupled to second screw 206B, that when rotated, causes the movement of second platform 207B on which the second product 204 is disposed, thereby exposing or retracting the second product 204 from the second chamber. The first turning knob 202A is just offset from the mid-section of dual-ended stick applicator 200 and the second turning knob is also proximate to, and just offset from, the midsection of dual-ended stick applicator 200. The first turning knob 202A is adjacent to second turning knob 202B and each turning knob rotates independent of the other.

The dual-ended stick applicator has a first opening with a corresponding removable cap dimensioned to fit securely over the first opening and a second opening with a corresponding removable cap dimensioned to fit securely over the second opening. In a further aspect, either of the first cap or the second cap or both have a flared end to support the dual ended container when stored upright or in a vertical orientation.

In the foregoing embodiment, the first product is a fast-acting but short duration anesthetic in semi-solid or hard gel form, the formulation suitable for application before applying a CGM or insulin pump to the skin. The anesthetic is formulated to dull the effectiveness of nociceptors. The invention 200 provides rapid onset of numbing or pain relief, but with a relatively brief duration of action. The anesthetic is one selected from Lidocaine, Benzocaine, Tetracaine, Prilocaine and EMLA Cream. The second product is an antiseptic in a semi-solid or hard gel form. The antiseptic is one selected from ethyl alcohol, isopropyl alcohol, povidone-iodine (Betadine), Chlorhexidine, Benzalkonium Chloride and Hydrogen Peroxide. Each product is formulated to dry quickly so as not to interfere with the skin adhesion, biocompatibility, and long-term wearability of the adhesive backing of the CGM or insulin pump.

In the foregoing embodiment, the dual-ended stick applicator 200 can be cylindrical or can be an elliptical cylinder. This term describes a three-dimensional geometric shape that resembles a traditional cylinder but has elliptical (oval-shaped) faces at each end instead of circular faces.

In a further embodiment of the dual-ended stick applicator 200, the first end has a first applicator for exposing a first product and the second end has a second applicator for exposing a second product.

The invention further a dual-ended stick applicator for semi-solid, gel-based antiseptic and anesthetic products. It offers convenience and versatility by combining two different topical treatments into a single, compact container. The invention is comprised of components and materials for the container, twist mechanisms, and dispensing ends to ensure compatibility with the semi-solid gel formulations. The twist mechanisms are designed to operate independently, allowing each end of the container to dispense the desired product separately.

Injection molding or blow molding processes are used to manufacture the container from plastic resin. The twist mechanisms are integrated into the center of the container during the molding process. The dispensing ends of the container are designed to accommodate the semi-solid or hard gel formulations, with openings for controlled dispensing. The semi-solid gel-based antiseptic and anesthetic products are filled into their respective compartments within the container. The twist mechanisms are assembled into the center of the container, allowing for independent operation of each end. The container is sealed to prevent leakage or contamination of the products. The dual-ended stick applicator is packaged in suitable packaging materials, such as blister packs or boxes, to protect it during transport and storage. Product labeling includes information such as usage instructions, ingredients, warnings, and branding elements.

The invention is a dual-ended stick applicator that provides convenient and targeted application of semi-solid, gel-based antiseptic and anesthetic products. It offers consumers a practical solution for addressing the pain associated with CGM sensor insertion and insulin pump canula insertion by providing fast, highly localized, short duration, pain relief. Various alternatives and permutations of the dual-ended container are within the scope of this invention. For example, a gel-based anesthetic, and talcum based drying agent can be the first product and second product, respectively.

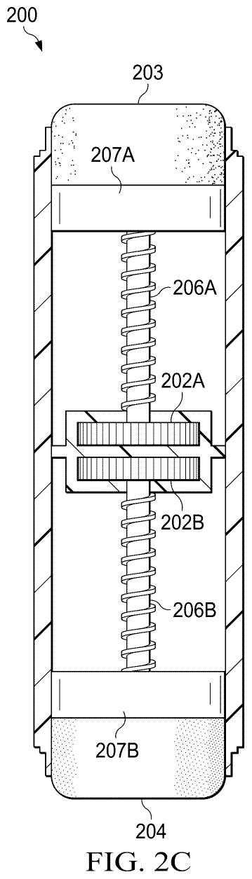

Now referring to FIG. 3A, there is seen a front view of a single-ended stick applicator 300 with a drying component 305 appurtenant to the outer container 302 with the cap 301 on. FIG. 3B is a front view of a single-ended stick applicator 300 with a drying component 305 appurtenant thereto with the cap 301 removed. FIG. 3C is a side view of a single-ended stick applicator 300 with a drying component 305 appurtenant thereto with the cap 301 removed. As seen therein, drying component 305 can be a compressed gas cartridge operable to release a compressed gas such as room temperature air, nitrogen, carbon dioxide, or argon through regulator 306 and nozzle 307 when activated using valve trigger 308. Regulator 306 can also include a heater/dryer portion to bring the compressed air to room temperature. The nozzle is operable to direct a stream of air to the skin on which an anesthetic or antiseptic has been applied so as to dry the skin so as to allow the medical adhesive of a CGM or insulin pump to remain secured to the skin.

Alternatively, drying component 305 appurtenant to the outer container 302 can be a squeezable rubber or silicone ball 305 that, when compressed and released, expels a stream of air through regulator 306 and nozzle 307 so as to dry the skin after application of product 304, which preferably is an anesthetic 304 or alternatively, an antiseptic.

As seen in FIGS. 3A, 3B and 3C, the invention also comprises an applicator that combines an anesthetic in a liquid, semi-solid or gel form with a drying component such as a compressed gas cartridge or squeezable silicone ball operable to direct or release a gas, such as room temperature air, nitrogen, carbon dioxide or argon toward the skin after application of the anesthetic so as to dry the skin without impacting the numbing effect of the anesthetic. One embodiment is a stick applicator with a sleek and ergonomic design that is comfortable to hold and easy to use. It is compact and lightweight for portability.

The anesthetic gel applicator portion has a chamber or reservoir to hold a gel such as Lidocaine or Benzocaine. The gel is dispensed through a nozzle or tip at the end of the stick applicator. The design allows for controlled and precise application of the gel to the desired area of the skin. In an aspect, the stick applicator has a separate compartment or mechanism for the compressed air portion. This compartment houses a small air cartridge or canister that can be activated to release a stream of compressed air. The stick applicator has an easy-to-use activation mechanism that allows the user to switch between dispensing the gel anesthetic, such as a Lidocaine gel, and releasing compressed air. This uses a button or switch located on the handle of the stick applicator.

The stick applicator includes safety features to prevent accidental activation or discharge of the compressed air from the drying component 305. This includes a locking mechanism or safety switch to prevent unintended activation. The compressed air drying component is designed to release a controlled stream of air onto the skin to facilitate drying of, e.g., a Lidocaine gel. The nozzle or outlet for the compressed air is positioned near the tip of the stick applicator for precise targeting of the drying airflow. The gel reservoir and compressed air cartridge are designed as replaceable or refillable components for convenience and hygiene purposes. The stick applicator is constructed from durable and hygienic materials that are compatible with the gel and compressed air.

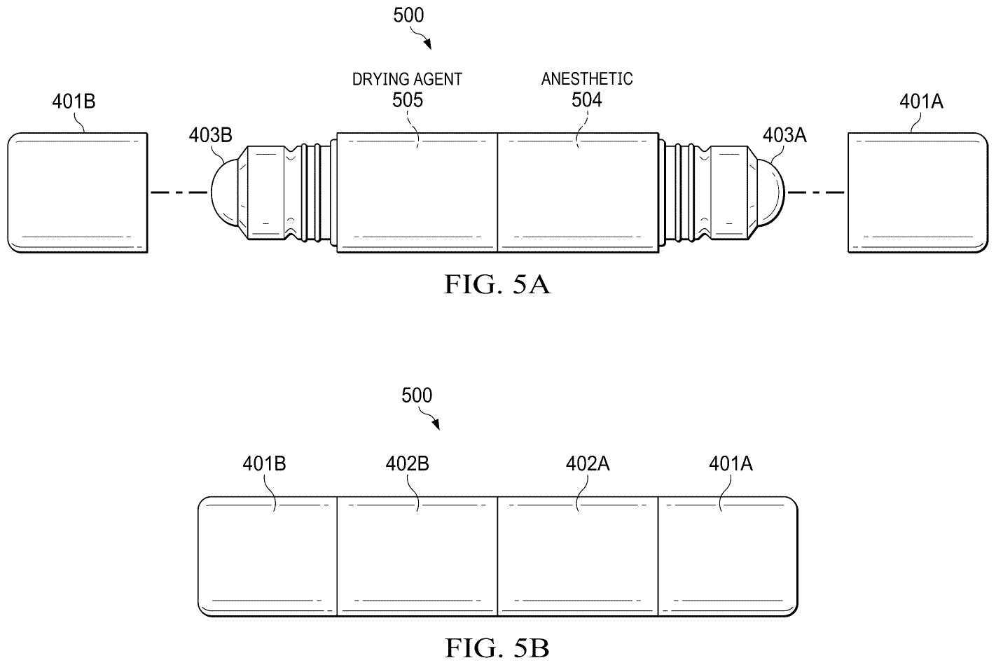

Regarding FIGS. 3A, 3B, 3C and 6, a compressed air cartridge stores air under high pressure inside a canister, which is then released when the nozzle is pressed. The cartridge contains a mixture of gases, typically compressed air or a blend of gases, which is stored under pressure. This compression is achieved by filling the canister with air and then sealing it tightly. The pressure inside the canister increases as more air is pumped in. The canister is equipped with a valve mechanism that controls the release of the compressed air. When the nozzle or trigger on the canister is pressed, it opens the valve, allowing the pressurized air to escape. As the valve opens, the high-pressure air inside the canister rushes out through the nozzle at high speed. This forceful release of air creates a powerful stream that can be directed towards the surface to be cleaned or dusted.

The high-velocity stream of air dries the surface of the skin. Compressed air cans use gases that are environmentally safe and non-toxic, such as compressed air or inert gases like nitrogen or argon. These gases do not leave behind residue or harmful chemicals, making them safe for use on sensitive electronic components. Once the compressed air can is emptied, it typically cannot be refilled or reused. The pressure inside the canister drops as the air is released, rendering it ineffective for further use. As a result, compressed air cans are typically considered single-use disposable items. However, there are canisters that allow reusable air canisters.

Air cartridges are small canisters filled with compressed gas. The manufacturing process begins with filling the cartridges with compressed gas. This is typically done in specialized facilities equipped with equipment capable of safely handling compressed gases. Once the cartridges are filled with the desired amount of gas, they are sealed to prevent leakage. This is usually achieved by crimping or sealing the opening of the cartridge with a cap or sealant. The cartridges are then packaged into individual units or multi-packs for distribution and sale. Packaging may include protective sleeves or covers to prevent accidental activation or damage during transport. To use an air cartridge, the threaded end of the air cartridge is then screwed onto the valve. The valve is then operated to release the compressed gas.

For applications where a gas is used in a cartridge to dry the skin, such as in aerosol sprays for drying skin or wound care products, compressed air or nitrogen are often the preferred gas. Compressed air and nitrogen are generally considered safer for skin contact. Both compressed air and nitrogen are non-toxic gases that are commonly used in various medical and industrial applications. They are generally well-tolerated by the skin and pose minimal risk of adverse effects. Compressed air and nitrogen provide a gentle and non-irritating means of drying the skin. They do not contain chemicals or additives that may cause irritation or allergic reactions. Compressed air and nitrogen can be used in a wide range of skin drying applications, including wound care, adhesive removal, and medical procedures. They offer versatility and flexibility in various healthcare settings. Overall, compressed air, nitrogen and argon are suitable options for use in cartridges designed to dry the skin.

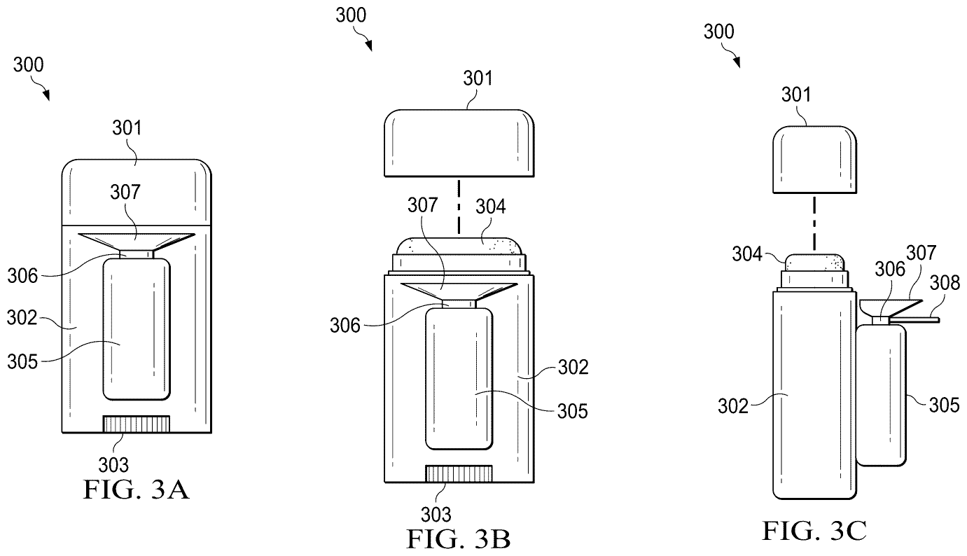

FIG. 4A is a view of a dual-ended rollerball applicator 400 having a viscous gel or liquid anesthetic product as a first product and a liquid or viscous gel drying agent product as the second product. In FIG. 4A, the caps 401A and 401B are removed. In FIG. 4B, the caps 401A and 401B are secured to the dual-ended rollerball applicator 400. The dual-ended rollerball applicator 400 has a first rollerball 403A and a second rollerball 403B, and a first container 402A and a second container 402B, first container 402A holding an anesthetic 404 and the second container 402B holding drying agent or antiseptic 405.

In one aspect, the invention 400 is a rollerball applicator in combination with a liquid formulation of a fast-acting but short duration anesthetic 404 with other inert ingredients suitable for application before applying a CGM or insulin pump to the skin. The invention 400 provides rapid onset of numbing or pain relief, but with a relatively brief duration of action. The fast-acting, short duration anesthetic can be one selected from Lidocaine, Benzocaine, Tetracaine, Prilocaine and EMLA Cream. Specifically, the anesthetic is formulated to dull the effectiveness of nociceptors. The liquid formulation is formulated to dry quickly so as not to interfere with the skin adhesion, biocompatibility, and long-term wearability of the adhesive backing of the CGM or insulin pump. Alternatively, the applicator can include a mechanism to cause the liquid formulation to be dried quickly so as not to interfere with the skin adhesion, biocompatibility, and long-term wearability of the adhesive backing of the CGM or insulin pump.

The invention 400 further comprises a rollerball applicator in combination with a liquid formulation of antiseptic and a liquid formulation of fast-acting but short duration anesthetic together in the applicator or in separate chambers of the applicator, the formulation suitable for application before applying a CGM or insulin pump to the skin. The antiseptic is one selected from ethyl alcohol, isopropyl alcohol, povidone-iodine (Betadine), Chlorhexidine, Benzalkonium Chloride and Hydrogen Peroxide and the anesthetic is one selected from Lidocaine, Benzocaine, Tetracaine, Prilocaine and EMLA Cream. Specifically, the anesthetic is formulated to dull the effectiveness of nociceptors. The liquid formulation is formulated to dry quickly so as not to interfere with the skin adhesion, biocompatibility, and long-term wearability of the adhesive backing of the CGM or insulin pump. Alternatively, the applicator can include a mechanism to cause the liquid formulation to be dried quickly so as not to interfere with the skin adhesion, biocompatibility, and long-term wearability of the adhesive backing of the CGM or insulin pump. As noted, the applicator 200 can have two chambers 402A, 402B with the anesthetic 404 contained within the first chamber 402A and the antiseptic 405 contained within the second chamber 402B. In an embodiment, the applicator can be designed to meter out a specific amount of each liquid serially. In a further embodiment, the applicator is designed to meter out a specific amount of each liquid concurrently. Further the applicator can include a third chamber to include a drying agent such as compressed air operable to assist in the quick drying of the liquid formulation.

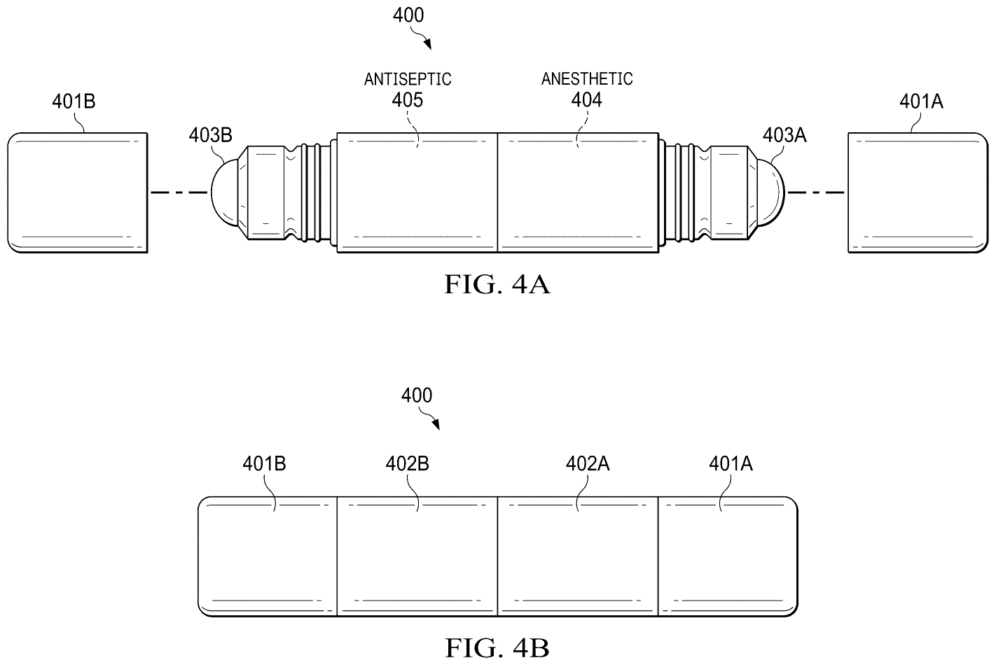

FIG. 5A is a view of a dual-ended rollerball applicator 500 with a first rollerball 403A and a second rollerball 403B having a viscous gel or liquid anesthetic product 504 as a first product and a liquid or viscous gel antiseptic product 505 as the second product with the caps 401A, 401B removed. FIG. 5B is a view of a dual-ended rollerball applicator 500 having a viscous gel or liquid anesthetic product 504 as a first product and a liquid or viscous gel antiseptic product 505 as the second product with the caps 401A, 401B on.

FIG. 6 is a view of a single-ended rollerball applicator 600 with cap 602, drying component 603 appurtenant thereto, the drying component 603 shown as a compressed gas cartridge with regulator and with the cap 602 removed. A liquid or viscous gel anesthetic product is contained within chamber 601 and operable to be dispensed via the rollerball. The invention 600 further comprises an applicator that combines an anesthetic in a liquid, semi-solid or gel form in a chamber 601 with a compressed gas portion 603 operable to release a gas, such as air, nitrogen, carbon dioxide or argon toward the skin after application of the anesthetic so as to dry the skin without impacting the numbing effect of the anesthetic. One embodiment is a rollerball applicator with a sleek and ergonomic design that is comfortable to hold and easy to use. It is compact and lightweight for portability.

The anesthetic liquid or gel rollerball applicator 600 has a chamber 601 or reservoir to hold a liquid or viscous gel such as Lidocaine. The liquid or gel is dispensed through the rollerball. The design allows for controlled and precise application of the gel to the desired area of the skin.

The rollerball applicator includes safety features to prevent accidental activation or discharge of the compressed air portion. This includes a locking mechanism or safety switch to prevent unintended activation. The compressed air portion is designed to release a controlled stream of air onto the skin to facilitate drying of the anesthetic. The nozzle or outlet for compressed air is positioned near the tip of the rollerball applicator for precise targeting of the drying airflow.

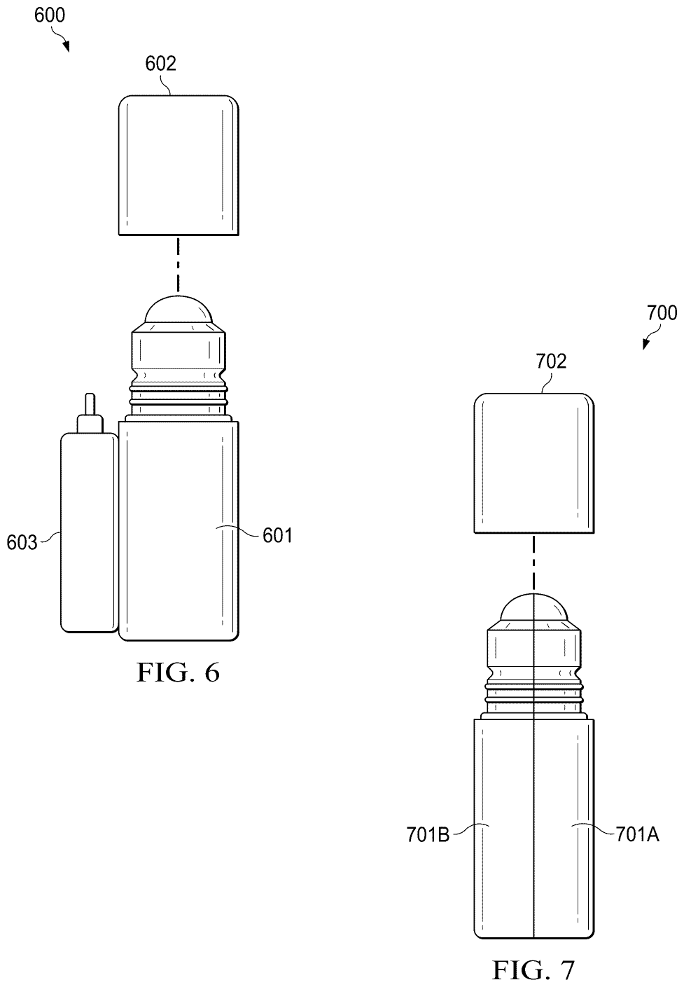

FIG. 7 is a view of a single-ended rollerball applicator 700 with cap 702 having a first chamber 701A and a second chamber 702B, one chamber for holding an anesthetic and the other chamber for holding an antiseptic or drying agent.

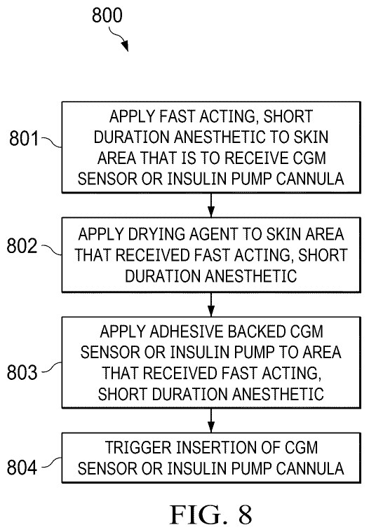

The invention further comprises the precursor method of applying a CGM or insulin pump to the skin by first applying an antiseptic, then applying an anesthetic, then applying a drying agent and/or drying the skin, then applying a CGM or insulin pump. FIG. 8 is a flow chart of a method 800 of the invention. The first step is to apply a fast acting, short duration anesthetic to skin area that is to receive CGM sensor or insulin pump canula 801. The next step is to apply drying agent to skin area that received fast acting, short duration anesthetic 802. The third step is to apply adhesive backed CGM sensor or insulin pump to area that received fast acting, short duration anesthetic 803. Trigger insertion of CGM sensor or insulin pump canula 804. The invention further comprises the method of using an anesthetic immediately prior to inserting a CGM sensor or insulin pump canula.

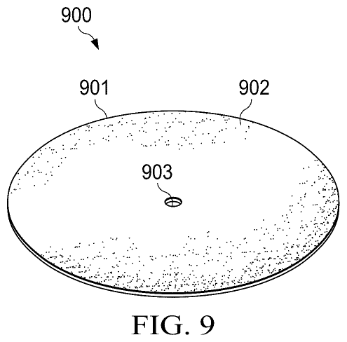

The invention further comprises an adhesive backing for use on a CGM or insulin pump, the adhesive backing infused with an anesthetic such as Lidocaine. FIG. 9 illustrates a further aspect of the invention 900 which is to apply an anesthetic formulation to all or a portion of the mesh fabric 902 of the adhesive patch 901 through which a CGM sensor or insulin pump canula traverses. Because the sensor or canula must pierce a portion of the adhesive patch 901 having the anesthetic, the spring mechanism in the CGM or insulin pump must have additional potential energy. In this way, when the adhesive backing of the CGM or insulin pump is applied to the skin, the anesthetic contacts the skin, numbing the area for the insertion of the CGM sensor or insulin pump canula. Alternatively, a small aperture 903 can be provided through which the sensor or canula is inserted into the skin while the anesthetic numbs the general area through which the sensor or canula is to enter the skin. The anesthetic will be fast acting but of short duration.

The invention further comprises a dual-ended stick applicator or a dual-ended rollerball applicator with a first end operable to provide or release an anesthetic or an antiseptic and a second end having a drying material. The dual-ended stick applicator includes a semi-solid or hard gel-based product disposed on a platform that is coupled to a twist up mechanism and operable to be exposed from the first end when a twist knob coupled as part of the twist mechanism is rotated. A cap is dimensioned to fit over the first end opening. An absorbent drying material is disposed on the second end operable to dry the anesthetic or antiseptic. Such an absorbent drying material can be a microfiber cloth, a terry cloth, a cotton pad, a linen fabric or a bamboo fabric. Microfiber cloths are known for their excellent absorbency and durability. They can be washed and reused multiple times without losing their effectiveness. Terry cloth is a highly absorbent fabric commonly used in towels. Cotton pads made from high-quality, tightly woven cotton can be reused many times while retaining their absorbency. Linen is a strong and durable natural fiber. Bamboo fabric is known for its absorbency and antimicrobial properties.

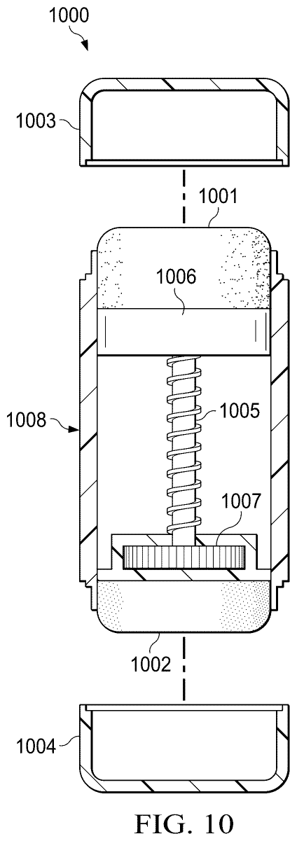

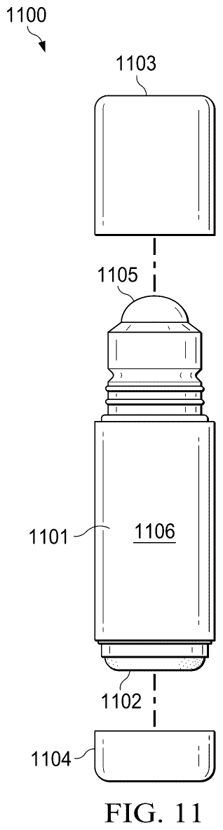

As seen in FIGS. 10 and 11, the invention further comprises a dual-ended stick applicator or a dual-ended rollerball applicator with a first end operable to provide or release an anesthetic or an antiseptic and a second end having a drying material.

FIG. 10 is a view of a dual-ended stick applicator 1000 having a first product, for example, a semi-solid or hard gel anesthetic or antiseptic product on a first end thereof and a drying material 1002 as the second product on a second end thereof, with the caps 1003, 1004 removed. The dual-ended stick applicator 1000 has a twist mechanism having a twist knob 1007 that when rotated, causes the movement of a first platform 1006 on which the first product is disposed, thereby exposing or retracting the first product from the container 1008 of invention 1000. The twist mechanism is coupled to a screw 1005 that is rotatably coupled to the platform 1006. The twist mechanism has a turning knob 1007 proximate the second end of the container 1008. Alternatively, the turning knob 1007 can be disposed in the center of the container 1008 or near the first end thereof. After the semi-solid product, such as an anesthetic or an antiseptic is applied to the skin, the area receiving the product can then be dried using the drying material 1102 so as to prepare the area for receiving a CGM sensor or insulin pump canula.

The dual-ended stick applicator 1000 includes a semi-solid or hard gel-based product disposed on a platform 1006 that is coupled to a twist up mechanism and operable to be exposed from the first end when a twist knob 1007 coupled to a screw 1005 as part of the twist mechanism is rotated. A cap 1003 is dimensioned to fit over the first end opening. An absorbent drying material 1002 is disposed on the second end operable to dry the anesthetic or antiseptic when it is swept across or is pressed, and the skin that received the anesthetic. Such an absorbent drying material 1002 can be any suitable absorbent product such as microfiber cloth, a terry cloth, gauze, a cotton pad, a linen fabric or a bamboo fabric. Alternatively, it can be a stack of one time usable per pad removable gauze, cotton or linen pads each having an impervious backing. Microfiber cloths are known for their excellent absorbency and durability. They can be reused multiple times without losing their effectiveness. Terry cloth is a highly absorbent fabric commonly used in towels. Cotton pads made from high-quality, tightly woven cotton can be reused many times while retaining their absorbency. Linen is a strong and durable natural fiber. Bamboo fabric is known for its absorbency and antimicrobial properties.

FIG. 11 is a view of a dual-ended rollerball applicator 1100 having a viscous gel or liquid anesthetic product as a first product 1106 and a drying material 1102 as the second product, with the caps 1103, 1104 removed. Rollerball 1105, when rolled over the skin, acts as a conduit of the first product from chamber 1101 to the skin. After the liquid is rolled onto the skin, the user then can dry the area of the skin using drying material 1102. This absorbent drying material 1102 can be any suitable absorbent product such as microfiber cloth, a terry cloth, gauze, a cotton pad, a linen fabric or a bamboo fabric. Alternatively, it can be a stack of one time usable per pad removable gauze, cotton or linen pads each having an impervious backing. Microfiber cloths are known for their excellent absorbency and durability. They can be reused multiple times without losing their effectiveness. Terry cloth is a highly absorbent fabric commonly used in towels. Cotton pads made from high-quality, tightly woven cotton can be reused many times while retaining their absorbency. Linen is a strong and durable natural fiber. Bamboo fabric is known for its absorbency and antimicrobial properties.

As discussed herein, the invention has a variety of embodiments and aspects. In one such embodiment, it is a stick applicator in combination with a semi-solid or hard gel anesthetic. In an aspect it is a solid, cylindrical product designed for topical application on the skin. Encased in a twist-up dispenser, it resembles a typical deodorant stick in appearance. As described herein, the preferred anesthetic for use with the invention is Lidocaine. Lidocaine formulations are also available in various forms, including liquids, gels, creams, and sprays. The viscosity and consistency of the Lidocaine formulation will determine its suitability for dispensing through an applicator. Lidocaine is a commonly used local anesthetic known for its rapid onset and effectiveness in providing local anesthesia. Lidocaine in the range of 2% to 35% by weight is the preferred anesthetic for use with the invention as it is known for its numbing properties, making it suitable for temporary relief of minor pain and discomfort associated with inserting a CGM sensor or insulin pump canula. Benzocaine formulations are commonly available in forms such as creams, gels, sprays which are specifically designed for topical application or localized anesthesia.

The anesthetic formulation would include suitable emollients and gelling agents to ensure a smooth and easy application, providing a comforting sensation upon contact with the skin. Its convenient stick format allows for precise and targeted application to the affected area without the need for additional tools or applicators.

The invention in the form of an anesthetic stick is compact and portable, making it ideal for on-the-go use and travel. Its twist-up design ensures hygienic dispensing and prevents product waste. The invention further features an oblong-shaped design, providing a unique twist on traditional stick applicators. With an elongated shape, the applicator fits comfortably in the hand, allowing for easy grip and precise application to the affected area. The oblong shape of the anesthetic stick applicator is characterized by its elongated form, featuring a longer length compared to its width. It is typically narrower and more slender than traditional cylindrical stick applicators, with gently rounded edges.

The invention further includes the process of making the invention. The first step of making the invention involves formulating the mixture for the anesthetic stick. This formulation would preferably include Lidocaine as the active ingredient, along with other components such as emollients, gelling agents, stabilizers, and possibly preservatives to achieve the desired consistency, stability, and skin compatibility. The ingredients are mixed in large mixing tanks. Depending on the specific formulation, heating may be required to ensure thorough blending and to achieve the desired consistency for the stick format. Once the mixture is thoroughly mixed and heated, if necessary, it is shaped into sticks. This can be done using extrusion equipment, where the mixture is forced through a shaping die to form the stick shape. Alternatively, it could be poured into molds if the formulation allows for it. After shaping, the sticks are cooled to solidify the formulation. Cooling may occur on a conveyor belt or in a controlled environment to ensure proper solidification and shape retention. Quality control procedures are implemented to ensure the final product meets safety, efficacy, and regulatory standards. This includes checks for appearance, texture and potency of the active ingredient. Additional tests may be conducted to ensure the product's stability over time. The final anesthetic sticks are packaged in appropriate containers, such as tubes or twist-up dispensers. Labels on the packaging contain important information regarding the active ingredient, usage instructions, warnings, and other relevant details.

Throughout the manufacturing process, strict quality control measures are implemented to ensure that the final product is safe, effective, and compliant with regulatory requirements for pharmaceutical products. The formulation of an anesthetic stick used for numbing an area of the skin would typically include several key ingredients, each serving a specific purpose in the formulation.

Lidocaine is the active ingredient responsible for providing the numbing effect. In an anesthetic stick, the proportion of Lidocaine would be in the range of between 2% to 35% by weight of the total formulation by weight. Emollients are ingredients that help soften and moisturize the skin, improving the spreadability of the product and enhancing skin comfort. Common emollients used in topical formulations include fatty acids, fatty alcohols, and oils such as mineral oil or petrolatum. Emollients typically make up a significant portion of the formulation, often ranging from 20% to 50% or more, depending on the desired texture and skin-feel of the product. Gelling agents are used to give the product its solid or semisolid consistency in stick form. Examples of gelling agents include waxes, polymers, and thickeners such as beeswax, carnauba wax, or cellulose derivatives. The proportion of gelling agents in the formulation can vary depending on the specific ingredients used and the desired consistency of the stick, but it typically ranges from 5% to 20%. Stabilizers are added to improve the stability and shelf life of the product by preventing degradation or separation of ingredients. Common stabilizers include antioxidants, chelating agents, and pH adjusters. The proportion of stabilizers is usually kept low, often less than 1% of the total formulation. Preservatives are included to prevent microbial growth and ensure product safety during storage and use. Examples of preservatives commonly used in topical formulations include parabens, phenoxyethanol, and benzyl alcohol. The proportion of preservatives is typically kept low, often less than 1% of the total formulation. Depending on the specific formulation and desired product characteristics, additional ingredients may be included, such as humectants, or skin-conditioning agents. The proportion of these additional ingredients varies depending on their intended function and compatibility with the overall formulation. The proportions of ingredients can vary depending on factors such as the specific formulation, regulatory requirements, and manufacturer preferences. Additionally, formulations may undergo testing and adjustments to optimize performance and ensure safety and efficacy.

Products with a gel or semi-solid texture that are designed to be smooth and easily spreadable on the skin typically have a viscosity ranging from 10,000 to 100,000 centipoise (cP) at room temperature. However, this range can vary depending on factors such as the specific formulation, intended use, and desired consistency of the products. Products with lower viscosity (closer to 10,000 cP) are lighter and more fluid, making them easier to spread and absorb into the skin quickly. Products with higher viscosity (closer to 100,000 cP) have a thicker consistency. The viscosity of a product can be influenced by various factors, including the types and concentrations of ingredients, emulsifiers, thickeners, and stabilizers used in the formulation. Manufacturers may also adjust the viscosity to achieve specific product characteristics and performance goals.

The invention is a device and product, comprising an applicator having a first end from which a first product is exposed, the first product being an anesthetic product; and a second end from which a second product is exposed. The first product is operable to reduce the pain associated with the insertion of a sensor or canula into the skin and the second product is operable to dry the area around which the first product was applied so as to allow the adhesion of an adhesive material to the skin. The sensor is a CGM sensor and the canula is an insulin pump canula. The device and product has a first twist-up mechanism for exposing the first product from the edge of the first end of the applicator and the anesthetic is in a semi-solid or hard gel form. The first twist-up mechanism includes a first turning knob that is accessible by a user. It also has a first threaded portion axially positioned within the applicator, the first turning knob securely coupled to the first threaded portion, a first platform having an integrated nut in the center thereof, the threaded portion being rotatably coupled to the integrated nut. The first product is disposed on the platform and having a bore therethrough to receive the threaded portion as the turning knob is rotated. The first twist-up mechanism being operable to raise or lower the first product so that it extends beyond the edge of the first end of the applicator when the first turning knob is rotated. The active ingredient in the anesthetic product is Lidocaine in the range of 2% to 35% of total weight of the anesthetic product.

The applicator includes a second twist-up mechanism independent of the first twist up mechanism and the second product is a drying agent. The applicator includes a second twist-up mechanism independent of the first twist up mechanism and the second product is a drying agent. The applicator includes a second twist-up mechanism independent of the first twist up mechanism, the second twist-up mechanism being operable to expose the second product beyond the edge of the second end of the applicator, wherein the drying agent is an antiseptic product in a semi-solid or hard gel form. The shape of the applicator is a substantially hollow ellipsoidal cuboid dimensioned to receive the first product within its walls, the first product in the form factor of an ellipsoidal cuboid. Alternatively, the shape of the applicator is a substantially hollow cylinder dimensioned to receive the first product within its walls, the first product also being that of a cylinder.

In a further aspect, the applicator includes a first rollerball mechanism having a chamber for retaining the first product and the anesthetic is contained in the first chamber in a liquid or viscous gel form. In a further aspect, the applicator includes a second rollerball mechanism independent of the first rollerball mechanism and the second product is a drying agent. The applicator has a first end cap dimensioned to fit over the first end of the applicator and a second end cap dimensioned to fit over the second end of the applicator.

The invention further comprises a device and product for reducing the pain associated with the insertion of a sensor or canula into the skin, comprising an applicator having a first end from which a first product is exposed for application on the skin, the first product being an anesthetic product wherein the applicator includes a twist-up mechanism, and the anesthetic is in a semi-solid or hard gel form.

The invention further includes a twist-up mechanism includes a turning knob that is accessible at an outer wall of the applicator; a threaded portion axially positioned within the applicator; the turning knob securely coupled to the threaded portion; and a platform having an integrated nut in the center thereof, the threaded portion being rotatably coupled to the integrated nut. The first product is positioned on the platform and having a bore therethrough to receive the threaded portion as the turning knob is rotated, the twist-up mechanism being operable to raise or lower the first product so that it extends beyond the edge of the first end of the applicator when the turning knob is rotated, wherein the active ingredient in the anesthetic product is Lidocaine in the range of 2% to 35% of total weight of the anesthetic product.

The invention is further a device and topical medical product for use when applying a bandage, medical adhesive, continuous glucose monitor (CGM) or insulin pump to an area of the skin, the device and topical medical product, having an applicator having a first end from which a first medical product is exposed, the first medical product being an anesthetic or antiseptic product; and the applicator having a second end from which an absorbent drying material is exposed. The invention further comprises the applicator having a first twist-up mechanism on which the first medical product is in a semi-solid or hard gel form is positioned. The first twist-up mechanism includes a first turning knob; a first threaded portion axially positioned within the applicator, the first turning knob securely coupled to the first threaded portion; a first platform having an integrated nut in the center thereof, the threaded portion being rotatably coupled to the integrated nut; the first product being positioned on the platform and having a bore therethrough to receive the threaded portion as the turning knob is rotated; and the first twist-up mechanism being operable to raise or lower the first product so that it extends beyond the edge of the first end of the applicator when the first turning knob is rotated. In an aspect, the active ingredient in the anesthetic product is Lidocaine in the range of 2% to 35% of total weight of the anesthetic product. The applicator includes an absorbent material at the second end of the applicator, wherein the absorbent material is one selected from the group consisting of microfiber cloth, cotton, gauze, terry cloth, linen, bamboo fabric and hemp fabric.

The invention is further a device and product for reducing the pain associated with the insertion of a sensor or canula into the skin, having an applicator having a first interface from which a first product is exposed, the first product being an anesthetic product; and the applicator further having a second interface from which a second product is exposed. The first interface is operable to expose a medicated stick and the second interface is a nozzle operable to direct a compressed gas. In a further aspect, the first interface is a rollerball operable to expose the first product and the second interface is a nozzle operable to direct a compressed gas. The invention further comprises a compressed gas cartridge being coupled to the applicator; a pressure regulator coupled to the cartridge; a valve coupled to the pressure regulator; and a nozzle coupled to the valve. In an aspect, the active ingredient in the anesthetic product is Lidocaine in the range of 2% to 35% of total weight of the anesthetic product. The invention further comprises a squeezable silicon ball being coupled to the applicator, a nozzle coupled to the squeezable silicon ball, the squeezable silicon ball operable, when squeezed, to direct air onto the skin so as to dry the first product.

The invention further comprises a device and product, a first product being in a semi-solid or hard gel form; a first twist-up mechanism for exposing the first product from the first end of the applicator; the first twist-up mechanism including a first turning knob that is accessible by a user; a first threaded portion axially positioned within the applicator, the first turning knob securely coupled to the first threaded portion; a first platform having an integrated nut in the center thereof, the threaded portion being rotatably coupled to the integrated nut; the first product being disposed on the platform and having a bore therethrough to receive the threaded portion as the turning knob is rotated; the first twist-up mechanism being operable to raise or lower the first product so that it extends beyond the edge of the first end of the applicator when the first turning knob is rotated. The second interface further is operable to expose a drying agent.

The invention further comprises a method for inserting a sensor or canula under the skin then applying an adhesive to the area around such insertion, comprising the steps of applying a fast acting, short duration anesthetic to skin area that is to receive a sensor or canula; applying a drying agent to skin area that received the fast acting, short duration anesthetic; and applying an adhesive backed device to area that received fast acting, short duration anesthetic. The invention further comprises the step of triggering the insertion of a sensor or cannula.

In such method invention, the adhesive backed device is a continuous glucose monitor (CGM) sensor or the adhesive backed device is an insulin pump. In such invention, the fast acting, short duration anesthetic is Lidocaine.

The invention further comprises a medical device to be affixed to the skin, comprising a processing unit having a member coupled thereto operable to be inserted into the skin; the processing unit having an adhesive backing having an aperture or membrane through which the member traverses; the adhesive backing having an anesthetic product around such aperture or membrane operable to contact and numb the skin prior to the insertion of the member. In such invention, the anesthetic product contains Lidocaine. In an aspect, the processing unit is operable to sense a blood glucose level and the member is a sensor. In a further aspect, the processing unit is operable to meter insulin and the member is a canula.

### Adhesion Testing Using Commercially Available Medical Grade Adhesive Bandage

### Time Between Application of Formulation and Drying Agent

### Concurrently

| Applied formulation | Drying Agent | Adhesion (Poor, Fair, Good) |
|---|---|---|
| Lidocaine 26% | Alcohol | Poor |
| | Talcum | Fair |
| | Compressed Air | Good |
| | None | Poor |
| Lidocaine 4% | Alcohol | Poor |
| | Talcum | Fair |
| | Compressed Air | Good |
| | None | Poor |

### Ten (10) Seconds

| Applied formulation | Drying Agent | Adhesion (Poor, Fair, Good) |
|---|---|---|
| Lidocaine 26% | Alcohol | Fair |
| | Talcum | Fair |
| | Compressed Air | Good |
| | None | Fair |
| Lidocaine 4% | Alcohol | Fair |
| | Talcum | Fair |
| | Compressed Air | Good |
| | None | Fair |

### Thirty (30) Seconds

| Applied formulation | Drying Agent | Adhesion (Poor, Fair, Good) |
|---|---|---|
| Lidocaine 26% | Alcohol | Good |
| | Talcum | Good |
| | Compressed Air | Good |
| | None | Fair |
| Lidocaine 4% | Alcohol | Good |
| | Talcum | Good |
| | Compressed Air | Good |
| | None | Fair |

### Sixty (60) Seconds

| Applied formulation | Drying Agent | Adhesion (Poor, Fair, Good) |
|---|---|---|
| Lidocaine 26% | Alcohol | Good |
| | Talcum | Good |
| | Compressed Air | Good |
| | None | Fair |
| Lidocaine 4% | Alcohol | Good |
| | Talcum | Good |
| | Compressed Air | Good |
| | None | Fair |

### Concurrently

| | | Adhesion |
|---|---|---|
| Applied formulation | Drying Agent | (Poor, Fair, Good) |
| Lidocaine 26% | Alcohol | Poor |
| and Antiseptic | | |
| together | | |
| | Talcum | Fair |
| | Compressed Air | Good |
| | None | Poor |
| Lidocaine 4% | Alcohol | Poor |
| and Antiseptic | | |
| together | | |
| | Talcum | Fair |
| | Compressed Air | Good |
| | None | Poor |

Ten (10) seconds

| | | Adhesion |
|---|---|---|
| Applied formulation | Drying Agent | (Poor, Fair, Good) |
| Lidocaine 26% | Alcohol | Fair |
| and Antiseptic | | |
| together | | |
| | Talcum | Fair |
| | Compressed Air | Good |
| | None | Fair |
| Lidocaine 4% | Alcohol | Fair |
| and Antiseptic | | |
| together | | |
| | Talcum | Fair |
| | Compressed Air | Good |
| | None | Fair |

### Thirty (30) Seconds

| | | Adhesion |
|---|---|---|
| Applied formulation | Drying Agent | (Poor, Fair, Good) |
| Lidocaine 26% | Alcohol | Good |
| and Antiseptic | | |
| together | | |
| | Talcum | Good |
| | Compressed Air | Good |
| | None | Fair |
| Lidocaine 4% | Alcohol | Good |
| and Antiseptic | | |
| together | | |
| | Talcum | Good |
| | Compressed Air | Good |
| | None | Fair |

### SIXTY (60) Seconds

| | | Adhesion |
|---|---|---|
| Applied formulation | Drying Agent | (Poor, Fair, Good) |
| Lidocaine 26% | Alcohol | Good |
| and Antiseptic | | |
| together | | |
| | Talcum | Good |
| | Compressed Air | Good |
| | None | Fair |
| Lidocaine 4% | Alcohol | Good |
| and Antiseptic | | |
| together | | |
| | Talcum | Good |
| | Compressed Air | Good |
| | None | Fair |

### Concurrently

| | | Adhesion |
|---|---|---|
| Applied formulation | Drying Agent | (Poor, Fair, Good) |
| Lidocaine 26% | Alcohol | Poor |
| and Antiseptic | | |
| separately | | |
| | Talcum | Fair |
| | Compressed Air | Good |
| | None | Poor |
| Lidocaine 4% | Alcohol | Poor |
| and Antiseptic | | |
| separately | | |
| | Talcum | Fair |
| | Compressed Air | Good |
| | None | Poor |

### Ten (10) Seconds

| | | Adhesion |
|---|---|---|
| Applied formulation | Drying Agent | (Poor, Fair, Good) |
| Lidocaine 26% | Alcohol | Fair |
| and Antiseptic | | |
| separately | | |
| | Talcum | Fair |
| | Compressed Air | Good |
| | None | Fair |
| Lidocaine 4% | Alcohol | Fair |
| and Antiseptic | | |
| separately | | |
| | Talcum | Fair |
| | Compressed Air | Good |
| | None | Fair |

### Thirty (30) Seconds

| | | Adhesion |
|---|---|---|
| Applied formulation | Drying Agent | (Poor, Fair, Good) |
| Lidocaine 26% | Alcohol | Good |
| and Antiseptic | | |
| separately | | |
| | Talcum | Good |
| | Compressed Air | Good |
| | None | Fair |
| Lidocaine 4% | Alcohol | Good |
| and Antiseptic | | |
| separately | | |
| | Talcum | Good |
| | Compressed Air | Good |
| | None | Fair |

### Sixty (60) Seconds

| | | Adhesion |
|---|---|---|
| Applied formulation | Drying Agent | (Poor, Fair, Good) |
| Lidocaine 26% | Alcohol | Good |
| and Antiseptic | | |
| separately | | |
| | Talcum | Good |
| | Compressed Air | Good |
| | None | Fair |
| Lidocaine 4% | Alcohol | Good |
| and Antiseptic | | |
| separately | | |
| | Talcum | Good |
| | Compressed Air | Good |
| | None | Fair |

### Pain Testing

Use of commercially available needle injector used for blood glucose testing

### Time Between Application of Formulation and Drying Agent

### Concurrently

| | | Pain Level (None, Low, Med, |
|---|---|---|
| Applied formulation | Drying Agent | High) |
| None | None | High |

| | | Pain Level (None, Low, Med, |
|---|---|---|
| Applied formulation | Drying Agent | High) |
| Lidocaine 26% | Alcohol | Med |
| | Talcum | Med |
| | Compressed Air | Med |
| | None | Med |
| Lidocaine 4% | Alcohol | High |
| | Talcum | High |
| | Compressed Air | High |
| | None | High |

### Ten (10) Seconds

| | | Pain Level (None, Low, Med, |
|---|---|---|
| Applied formulation | Drying Agent | High) |
| Lidocaine 26% | Alcohol | Low |
| | Talcum | Low |
| | Compressed Air | Low |
| | None | Low |
| Lidocaine 4% | Alcohol | Med |
| | Talcum | Med |
| | Compressed Air | Med |
| | None | Med |

### Thirty (30) Seconds

| | | Pain Level (None, Low, Med, |
|---|---|---|
| Applied formulation | Drying Agent | High) |
| Lidocaine 26% | Alcohol | None |
| | Talcum | None |
| | Compressed Air | None |
| | None | None |
| Lidocaine 4% | Alcohol | Low |
| | Talcum | Low |
| | Compressed Air | Low |
| | None | Low |

### Sixty (60) Seconds

| | | Pain Level (None, Low, Med, |
|---|---|---|
| Applied formulation | Drying Agent | High) |
| Lidocaine 26% | Alcohol | None |
| | Talcum | None |
| | Compressed Air | None |
| | None | None |
| Lidocaine 4% | Alcohol | Low |
| | Talcum | Low |
| | Compressed Air | Low |
| | None | Low |

Even though numerous characteristics and advantages of the present invention have been set forth in the foregoing description, the disclosure is illustrative only and changes may be made within the principles of the invention to the full extent indicated by the broad general meaning of the terms used herein. Various alterations, modifications and substitutions can be made to the disclosed invention without departing in any way from the spirit and scope of the invention.

The invention claimed is:

1. A device and product, comprising:
an applicator having a first end from which a first product is exposed, the first product being an anesthetic product; and
a second end from which a second product is exposed.

2. The device and product of claim 1, wherein the first product is operable to reduce the pain associated with the insertion of a sensor or canula into the skin and the second product is operable to dry the area around which the first product was applied so as to allow the adhesion of an adhesive material to the skin.

3. The device and product of claim 2, wherein the sensor is a CGM sensor and the canula is an insulin pump canula.

4. The device and product of claim 1, further comprising a first twist-up mechanism for exposing the first product from the edge of the first end of the applicator and the anesthetic is in a semi-solid or hard gel form.

5. The device and product of claim 4, wherein the first twist-up mechanism includes a first turning knob that is accessible by a user;
a first threaded portion axially positioned within the applicator, the first turning knob securely coupled to the first threaded portion;
a first platform having an integrated nut in the center thereof, the threaded portion being rotatably coupled to the integrated nut;
the first product being disposed on the platform and having a bore therethrough to receive the threaded portion as the turning knob is rotated;
the first twist-up mechanism being operable to raise or lower the first product so that it extends beyond the edge of the first end of the applicator when the first turning knob is rotated.

6. The device and product of claim 1, wherein the active ingredient in the anesthetic product is Lidocaine in the range of 2% to 35% of total weight of the anesthetic product.

7. The device and product of claim 1, wherein the applicator includes a second twist-up mechanism independent of the first twist up mechanism and the second product is a drying agent.

8. The device and product of claim 1, wherein the applicator includes a second twist-up mechanism independent of the first twist up mechanism, the second twist-up mechanism being operable to expose the second product beyond the edge of the second end of the applicator, wherein the drying agent is an antiseptic product in a semi-solid or hard gel form.

9. The device and product of claim 1, wherein the shape of the applicator is a substantially hollow ellipsoidal cuboid dimensioned to receive the first product within its walls, the first product in the form factor of an ellipsoidal cuboid.

10. The device and product of claim 1, wherein the shape of the applicator is a substantially hollow cylinder dimensioned to receive the first product within its walls, the first product also being that of a cylinder.

11. A device for reducing the pain associated with the insertion of a sensor or canula into the skin, comprising:
the device having an applicator on a first end thereof from which a first product is exposed for application on the skin, the first product being an anesthetic product wherein the applicator includes a twist-up mechanism and the anesthetic is in a semi-solid or hard gel form, wherein the twist-up mechanism includes a turning knob that is accessible at an outer wall of the applicator;
a threaded portion axially positioned within the applicator; the turning knob securely coupled to the threaded portion;
a platform having an integrated nut in the center thereof, the threaded portion being rotatably coupled to the integrated nut;
the first product being disposed on the platform and having a bore therethrough to receive the threaded portion as the turning knob is rotated, the twist-up mechanism being operable to raise or lower the first product so that it extends beyond the edge of the first end of the applicator when the turning knob is rotated, wherein the active ingredient in the anesthetic product is Lidocaine in the range of 2% to 35% of total weight of the anesthetic product;
the device having a drying material extending from the second end thereof, the second end being opposite the first end.

12. The device for reducing the pain associated with the insertion of a sensor or canula into the skin of claim 11, wherein the active ingredient in the anesthetic product is Lidocaine in the range of 2% to 35% of total weight of the anesthetic product.

13. The device for reducing the pain associated with the insertion of a sensor or canula into the skin of claim 11, wherein the drying material at the second end of the applicator is one selected from the group consisting of microfiber cloth, cotton, gauze, terry cloth, linen, bamboo fabric and hemp fabric.

14. A device and topical medical product for use prior to applying a bandage, medical adhesive, continuous glucose monitor (CGM) or insulin pump to an area of the skin, the device and topical medical product, comprising:
an applicator having a first end from which a first medical product is exposed, the first medical product being an anesthetic or antiseptic product; and
the applicator having a second end opposite from the first end thereof and from which an absorbent drying material is exposed.

15. The device and topical medical product of claim 14, further comprising the applicator having a first twist-up mechanism on which the first medical product is in a semi-solid or hard gel form is positioned.

16. The device and topical medical product of claim 15, wherein the first twist-up mechanism includes a first turning knob;
a first threaded portion axially positioned within the applicator, the first turning knob securely coupled to the first threaded portion;
a first platform having an integrated nut in the center thereof, the threaded portion being rotatably coupled to the integrated nut;
the first product being positioned on the platform and having a bore therethrough to receive the threaded portion as the turning knob is rotated; and
the first twist-up mechanism being operable to raise or lower the first product so that it extends beyond the edge of the first end of the applicator when the first turning knob is rotated.

17. The device and topical medical product of claim 14, wherein the active ingredient in the anesthetic product is Lidocaine in the range of 2% to 35% of total weight of the anesthetic product.

18. The device and topical medical product of claim 14, wherein the active ingredient in the anesthetic product is Benzocaine in the range of 2% to 35% of total weight of the anesthetic product.

19. The device and topical medical product of claim 14, wherein the absorbent drying material at the second end of the applicator is one selected from the group consisting of microfiber cloth, cotton, gauze, terry cloth, linen, bamboo fabric and hemp fabric.

20. The device and topical medical product of claim 14, wherein the absorbent drying material at the second end of the applicator is compacted microfiber cloth.
